(19) United States
(12) Patent Application Publication
(10) Pub. No.: US 2014/0336557 A1
(43) Pub. Date: Nov. 13, 2014

(54) BIOPOLYMER MULTI-LAYER MULTI-FUNCTIONAL MEDICAL DRESSING AND METHOD OF MAKING SAME

(71) Applicant: Biovation II, LLC, Boothbay, ME (US)

(72) Inventors: Kerem Durdag, Scarborough, ME (US); Valerie Gunn, Cape Neddick, ME (US); Brittany Paul, Old Orchard Beach, ME (US); Robert Hamlyn, Newcastle, ME (US); Brian Pendleton, Newcastle, ME (US); Marc Etchells, West Hampton, MA (US)

(21) Appl. No.: 14/267,161

(22) Filed: May 1, 2014

Related U.S. Application Data
(60) Provisional application No. 61/821,826, filed on May 10, 2013.

(51) Int. Cl. A61L 15/64; A61L 15/44; A61F 13/02; A61L 15/18

(52) U.S. Cl. CPC A61L 15/64; A61L 15/18; A61L 15/44; A61F 13/0216; A61F 2013/00323
USPC 602/48; 604/319

(57) ABSTRACT

The Technology described herein applies to medical dressings designed to heal wounds in the area of advanced wound care, inclusive of Negative Pressure Wound Therapy (NPWT), and describes novel wound healing absorbent scaffolds and dressing based on natural and naturally-derived material and fibers, preferentially poly (lactic) acid fibers and alginate materials.

### FIELD OF INVENTION

This invention relates generally to advanced wound healing, and the use of non-collapsible, scaffold devices, to heal severe wounds that do not respond to conventional treatment. The present invention relates more specifically to cross-linked biopolymer medical dressings affording the combined best features of the current standard absorbent dressings such as foam, alginate, hydrocolloid and hydrogel. Further, the improvements described herein comprise increased fluid uptake and retention, comfort, conformability and ease of removal from the wound bed.

### BACKGROUND OF THE INVENTION

There are many wounds that do not heal by conventional techniques. Typically, such wounds have large surface areas and/or deep wound beds where conventional wound closure techniques do not work. In such wounds, re-epithelialization and subsequent tissue migration and closure are generally compromised. Large surface area wounds, such as burns, diabetic ulcers and sores are also prone to infection and have an abundance of necrotic tissue. Techniques such as Negative Pressure Wound Therapy (NPWT), foam, hydrocolloid and hydrogel products are widely used to help heal these wounds.

Foam and current NPWT devices use some form of porous structure that is placed in the wound bed to allow the flow of air and wound exudate and provide a means to prevent the collapse of a top non-porous sheet or device on the wound as the pressure is reduced. Typically, open-cell foams or gauze pads are employed, each one problematic. In the former case, the open-cell foams are synthetic, not resorbable in the body and have sharp edges that may cause point-pressure contact in the wound. In the latter case, the gauze is also non-resorbable and may not always provide enough rigidity such that more elaborate devices need to be constructed to overcome the gauze's propensity to collapse under reduced pressure. In each case, the structure contacts the wound and may adhere to the wound, causing complications. Additionally, both often deposit small fibers or particles into the wound as foreign bodies. Often, in practice, a non-adherent layer of petroleum jelly is applied to the wound-contact surface, which introduces another foreign material and complicates the clinical practice of NPWT and foam dressings.

A mainstay of wound management in burn patients, who are especially susceptible to infection, uses topical creams or solutions containing silver (e.g., silver sulfadiazine). However, they have the disadvantage of staining the skin and have known toxicity. In addition, these techniques require frequent removal and reapplication to control the development of pseudoeschar. This is time consuming for professionals and painful for patients. A very wide range of antimicrobial dressings containing silver either incorporated within or applied to the dressing are now available for clinical use. This new class of dressings is designed to provide the antimicrobial activity of topical silver in a more convenient application. However, the various dressings differ considerably in the nature of their silver content and in their physical and chemical properties.

UK patent application GB2195225A by Vacutec Ltd. describes a subatmospheric pressure source being connected to a wound care assembly via a tube attached to an airtight assembly comprised of plastic sheet materials which extend beyond the wound area, seal around the skin epidermis and enclose a porous layer of felt defined as nonwoven, woolen fibers that resides in the wound bed. GB2195225A does not mention the use of non-woolen fibers and does not discuss re-adsorption capabilities of the wound contact material.

U.S. Pat. No. 5,636,643, assigned to Wake Forest University, describes a very similar device and identical end use. Instead of a felt nonwoven structure, U.S. Pat. No. 5,636,643 employs a screen which may be formed of a rigid or semi-rigid perforated polymer surgical mesh exemplified as Prolene® mesh. Alternatively disclosed is a section of honeycombed polyethylene sheet that may be cut to a suitable size and shape to overlie the wound. The porous layer may be a foam screen.

U.S. Pat. No. 5,645,081, assigned to Wake Forest University, describes a similar device and identical end use to U.S. Pat. No. 5,636,643. Specifically, their porous layer material, which is used to prevent overgrowth of tissue in the wound area, can be cut to fit the wound and is porous so that oxygen can react with the wound. They specify the use of a spongy polymeric foam material or a honeycombed polyethylene sheet.

U.S. Pat. No. 7,198,046, assigned to Wake Forest University Health Sciences, details a negative pressure wound healing apparatus that uses a porous layer of open-cell foam or a rigid porous support screen placed between the wound cover and the wound bed. This patent also describes open-cell foam for placement in the wound.

U.S. Pat. No. 7,776,028, assigned to Blue Sky Medical Group Incorporated, details a reduced pressure, treatment appliance and focuses on a cupped overlay apparatus that has membranes fitted over the wound and provides for ports to apply vacuum. The patent also mentions a wound packing material (porous layer) consisting of absorbent dressings, antiseptic dressings, non-adherent dressings, water dressings, or combinations of such dressings. It also mentions using gauze and cotton to pack the wound and mentions an absorbable matrix adapted to encourage growth of tissue in the wound area wherein the absorbable matrix is collagen.

U.S. Pat. No. 8,084,663 B2, assigned to KCl Licensing Incorporated, describes a vacuum therapy appliance, wherein the wound dressing has a hydrophobic or biodegradable wound contact layer and one or more absorbent layers for absorbing fluid from the wound. The absorbent layers can be quilted with patches containing desiccant or absorbent materials. The patent also mentions the wound dressing may allow fluid to pass through to the suction member. A semi-permeable cover is provided which allows the wound to breathe while protecting the wound from such undesirable substances as bacteria, viruses, and/or exogenous fluids. It also mentions the capability to incorporate sensors into the wound dressing to monitor the physiological parameters of the wound such as oxygen saturation, blood glucose level and serous fluid turbidity to name a few. The patent further mentions that medicaments may be introduced into the wound through the wound dressing. The patent mentions a hydrophobic and/or biodegradable layer at the wound interface. The patent mentions potentially anti-infective characteristics of brewer's yeast extract which is used in one example to fabricate the base layer

Wound repair requires the coordinated control over many different biological processes including but not limited to inflammation, angiogenesis, cellular remodeling, the development of granulation tissue (re-epithelialization) and, most significantly, infection control and prevention. The increasing incidence of wound microbial bioburden (in both the planktonic and biofilm phenotypic states) as well as increases in both bacterial virulence and pathogenicity will significantly impede the wound healing process (Percival S L, Thomas J G, Williams D W. Int. Wound J 2010; 7: 169-75.). In addition, many micro-organisms produce toxins, enzymes, and pro-inflammatory cytokines which are also detrimental to wound healing (Percival S L, Cochrane C A. In Percival S L, Cutting K, editors. Microbiology of Wounds. CRC Press: New York, 2010).

The prevention of infection or the justification for the use of antimicrobials in the management of chronic wound infections must demonstrate activity against the microorganisms in both their planktonic and biofilm states because each of these phenotypes may exhibit a significantly different tolerance toward antimicrobials. Since biofilms are generally associated with delayed wound healing, it is of paramount importance to demonstrate the efficacy of antimicrobial activity against wound biofilms (Wolcott R D, et al. J Wound Care 2010; 19: 45-6, 48-50, 52-53.).

Antimicrobials, such as ionic silver, offer a proven ability to inhibit the growth of and to kill microorganisms when the silver is present within or on the surface of the wound dressing (Wolcott R D, et al. J Wound Care 2008; 17: 502-8; Percival S L, Dowd S. In Percival S L, Cutting K, editors. Microbiology of Wounds. CRC Press: New York, 2010; Beele H, Meuleneire F, Nahuys M, Percival S L. Int. Wound J 2010; 7: 262-70.). Silver impregnated wound dressings have also been shown to be effective against antibiotic resistant and “silver” resistant bacteria (Percival S L, Bowler P, and Woods E H. Wound Repair Regen 2008; 16: 52-7). In addition, these ionic silver impregnated wound dressings have been linked to observations of enhanced wound healing (Miller C N, et al. Wound Repair Regen 2010; 18: 359-67.). Silver Alginate fiber wound dressings, in particular, have demonstrated the ability to adjust wound exudate levels and to maintain an effective level of antimicrobial activity at the wound-dressing interface as well as within the wound dressing itself (Bradford C, Freeman R, Percival S L. J. Amer. Col. Certif. Wound Spec. 2009; 1: 117-20.).

The two most important functions of surgical or wound dressings are 1) the ability to absorb and hold fluid and 2) the ability to quickly wick and transfer wound exudate away from the wound site. In order for the wound to heal properly, the wound bed must be kept moist. Therefore the wicking and transfer of the wound exudate must be achieved without desiccating the wound bed. The wound dressing should be soft, comfortable and conforming to the wound to ensure optimal performance and maximum patient compliance. In addition, the dressing should release easily from the wound so that the removal of the dressing does not damage the fragile, newly formed tissue.

Alginate fiber dressings (biodegradable dressings derived from seaweed) produce a warm, moist environment for healing wounds including chronic, infected ulcer wounds. The fibers react with wound exudate to form an absorbent gel, which keeps the wound moist. Changing this type of dressing includes washing the saturated gel out of the wound with saline solution so as not to disturb the newly formed tissue.

Calcium alginate fibers, produced by a wet-spinning process to make a non-woven dressing by first forming an ion-active gel over the wound site, react with the sodium ions in the wound exudate to assist wound healing. This ion exchange of calcium ions for sodium ions present in the wound exudate forms a gel which functions as a dressing for wound moisture management. The calcium ions introduced into the wound during the exchange will then be available to encourage clot formation. Additionally, the present invention also contemplates the use of magnesium ions, chromium ions and zinc ions as suitable for use in the present invention.

It has long been known that alginates which have been woven into a gauze or in the form of loose absorbent cotton-like wool as described by U.S. Pat. No. 2,512,616, are particularly useful as surgical dressings and/or wound packing materials as disclosed by U.S. Pat. No. 3,879,168.

In addition, U.S. Pat. No. 4,837,024 details a glycosaminoglycan (alginate)—collagen complex for enhanced wound healing. Glycosaminoglycans, which also include keratins, chondroitins and hyaluronans, are chemotactic for fibroblasts and epithelial cells, as described above in [0010], and promote vascularization as well as providing a favorable environment for the cells to participate in the wound healing process.

Non-fibrous alginate wound dressings are also well known, as evidenced by U.S. Pat. No. 4,393,080, which discloses a gel wound dressing that is formed from a water soluble hydrogel, an alkali metal alginate and glycerin. Further, U.S. Pat. No. 4,948,575 describes a dimensionally stable water insoluble alginate hydrogel foam wound dressing that is formed in place, either on the wound surface or in the wound cavity, as it gels from a reactive composition.

Biopolymer gelled composites, particularly cross-linked alginate gels as described in U.S. Pat. No. 7,674,837 B2, invented by and assigned to FMC Biopolymer AS, mentions potential application for medical use including, but not limited to, wound dressings, controlled sustained release delivery systems and bioabsorbable implants. Historically, these biopolymer gels have proven to be brittle and difficult to handle as well as both difficult and expensive to manufacture often requiring expensive equipment such as freeze driers. FMC Biopolymer AS made significant improvements to the existing technology through developments in the control of bubble generation, bubble size and gelling rate resulting in the production of mechanically homogeneous gelled and cured polymeric bubbles. In addition, these technology advancements eliminated the need for the aforementioned expensive drying equipment previously required to manufacture said materials. This patent mentions all potential uses of the improved process for making cured biopolymer bubbles or gelled composites including, but not limited to, food applications, personal care applications such as oral hygiene and cosmetic use, wound dressing materials, controlled release delivery systems, cell culture, barrier material for preventing tissue adherence and bioabsorbable implants. The patent also mentions the potential incorporation of all of the standard features and benefits of traditional wound dressings such as antimicrobial agents, bioactive materials for enhanced wound healing, medicines and other agents to be delivered in a controlled release manner by or through the dressing into the wound. The examples cited are comprised of formulation variations to achieve numerous specific biopolymer gelled composite properties as well as the process conditions required to best manufacture these unique materials. The claims are well supported by the examples and are specific to formulations and process parameters and do not reflect the specifics of any application, in particular those for a wound dressing with or without antimicrobial or other bioactive efficacy. Therefore, we incorporate the teachings of U.S. Pat. No. 7,674,837 B2 by reference and with permission of the inventors, herein within this patent as part of its application, with some significant and unique improvements, as a novel wound dressing/Controlled Sustained Release (CSR) delivery system.

### SUMMARY OF THE INVENTION

The present invention provides new and non-obvious technology directed towards medical dressings to heal wounds in the area of advanced wound care, inclusive of Negative Pressure Wound Therapy (NPWT), and describes novel wound healing absorbent scaffolds and dressings based on natural and naturally-derived material and fibers, preferentially poly (lactic) acid fibers and alginate material that possess distinct advantages over the various foams and gauze now employed in the art. Such a new wound healing absorbent dressing uses fibers and materials that have inherently low bioburden, have the capability to deliver antimicrobial agents for infection control properties, have full bio-compatibility, are completely non-toxic and resorbable in the body, can selectively degrade, are non-adherent to body tissue, and can have hydrophilic and hydrophobic surfaces. This novel absorbent dressing has other advantages such as: high mechanical wet strength in the wound bed, easy conformability to the wound, and a pliable and flexible structure with no sharp edges that can cause pressure and stress in the wound. Even in the event the dressing is cut to shape, there are no sharp edges or material that can disassociate from the dressing and reside in the wound causing a secondary infection site. The fibers are single continuous filaments and are both bio-resorbable and biocompatible. This monofilament design of the present invention has the advantage of minimizing breakage, selectively controlling the release of active antimicrobial ingredients, creating a wound scaffold and allowing the manufacture of advanced wound healing platforms. In addition, this monofilament structure may be used as an absorbent media and may be established as a continuous sheet or filament from, but not limited to, a minimum diameter of 1 micron to a maximum diameter of 100 microns, affording stand-alone structures.

The gel cast composite and active layer are comprised of biopolymer materials commonly available in nature including but not limited to glucosamino glycans, polysaccharides, starches, cellulosics, et al. The cross-linked, biopolymer gelled composite may be coated with an excipient (active layer) as a Controlled Sustained Release (CSR) delivery system, where one or more of the ingredients are anti-inflammatory agents, antibacterial and antifungal agents, antibiotics, antiseptics, agents for cancer treatment, Nitric Oxide generating materials for the treatment of chronic wounds, fibroblast and epithelial cell chemotactic agents, hyaluronans, humectants and other medicaments and/or cosmetic agents known in the art.

This invention defines a method for forming a moist, cross-linked gelled biopolymer composite to satisfy a still existing need for a soft, pliable, highly absorbent dressing to deliver moisture and other healing and anti-infective materials to low exudating and burn injury wounds. This embodiment absorbs and tightly holds moisture at temperatures below 35° C. and releases moisture in a controlled fashion at or above 35° C. The release of moisture occurs when the dressing comes in contact with the skin allowing for imminent halting of the burning process followed by evaporative cooling which in combination with anti-infective and other healing agents creates a favorable environment for healing of the burn injury wound.

In one aspect, the invention comprises the incorporation of antimicrobial agents including but not limited to silver, silver salts, iodine, chlorohexidine esters and chitosan within or on the surface of the gelled biopolymer composite dressing. Another aspect of the present invention, while also inclusive of the aforementioned antimicrobial aspect, comprises a controlled and sustained release delivery system of bioactive agents such as hyaluronans which are chemotactic for fibroblasts and epithelial cells, anti-inflammatory agents, antibacterial and antifungal agents, antibiotics, antiseptics, agents for cancer treatment, Nitric Oxide generating materials (natural and synthetic) for the treatment of the chronic wounds resultant from cancer treatments, diabetes, pressure ulcers, vascular insufficiencies and other wounds associated with advanced age and suppressed immune capacity. These aspects may be implemented as primary wound dressings or as a component of a vacuum assisted wound care apparatus such as commonly used in Negative Pressure Wound Therapy (NPWT). Independent of, or in concert with, the aforementioned embodiments, an additional aspect of this invention comprises the incorporation of a cosmetic agent either dispersed within the cross-linked biopolymer gelled composite or within an active coating applied to the surface of said composite. The cosmetic agent of the present invention may be cosmetics, drugs, quasi-drugs or medicines, which traditionally are applied topically in cream or lotion form. In this invention, we include in particular as cosmetic agents those active ingredients which are used in the cleaning and care of skin. Ingredients of this type are employed to maintain healthy skin condition, protect skin from damaging environmental conditions such as excessive solar/UV radiation, protection of skin from laundry and cleansing agents as well as other environmental stress such as dust and emissions. Natural oils (including but not limited to avocado oil, coconut oil and olive oil, and other generally recognized as safe (GRAS) vegetable oil or sustainable sourced oils), vitamins, collagens, oligoproteins, collagen-hydrolysates, humectants such as hyaluronans, sorbitol, glycerin, and known UV filtering or inhibiting substances (agents; for example to methyl paraben and propyl paraben) are also included as cosmetic agents, all of which are known to one of ordinary skill in the art.

An additional aspect of the invention defines a method for forming a moist, cross-linked gelled biopolymer composite to satisfy a still existing need for a soft, pliable, highly absorbent dressing to deliver moisture and other healing and anti-infective materials to low exudating and burn injury wounds. This embodiment absorbs and tightly holds moisture at temperatures below 35° C. and releases moisture in a controlled fashion at or above 35° C. This formulation will, for example, comprise methyl cellulose, poly (vinylcaprolactam), hydroxypropyl cellulose (HPC) and/or poly (N-isopropyl acrylamide) within, between or on the surface of the cross-linked biopolymer gelled composite. The release of moisture occurs when the dressing comes in contact with the skin allowing for imminent quenching of the burning process followed by evaporative cooling which in combination with anti-infective and other healing agents creates a favorable environment for healing of the burn injury wound.

In one embodiment, the present invention contemplates a medical dressing comprising a biopolymer layered structure, the layered structure comprising: a biodegradable, bioresorbable layer comprising a plurality of biodegradable, bioresorbable fibers, wherein the fibers are oriented to provide compression resistance and maintain paths for liquid-flow and air-flow, and a bioresorbable, biodegradable hydrophilic surface coating on a substantial number of the fibers; the fibers incorporating one or more bioactive agents.

The present invention further contemplates that the layered structure may comprise one or more natural fibers selected from the group consisting of cotton, bamboo and sisal and that the layered structure may comprise one or more synthetic fibers selected from polylactide, polyglycolide, poly-L-lactide, poly-DL-lactide and poly caprolactone.

The present invention further contemplates that the bioresorbable hydrophilic surface coating is on a substantial number of the fibers located proximate to other layers of the medical dressing and that the bioresorbable hydrophilic surface coating may comprise one or more of cellulose, alginate, gums, starch, chitosan, ethylene glycol, poly-oxethylene and polylactic acid.

The present invention further contemplates that each of the fibers in the plurality of fibers has a diameter of approximately 1 μM to 1 mm or, more preferably, each of the fibers in the plurality of fibers has a diameter of approximately 5 to 100 microns. Further, the present invention contemplates that the diameter of the fibers is selected to provide a desired compression resistance between a range of 0% and 75%, 0% and 50% and between 5% and 30%.

The present invention further contemplates that the fibers of the medical dressing are processed by one or more of being cut into a staple of selected length, carded, air-layered, needle-punched, vertically lapped, spirally wound, thermally bonded, or ultrasonically bonded.

The present invention further contemplates that the bioactive agent of the medical dressing is an antimicrobial agent and that the antimicrobial agent may comprise a silver-species. Further, the bioactive agent may be a component of or applied to one or more of the fibers and/or the surface coating.

The present invention further contemplates that the medical dressing further comprises: a semi-permeable layer over-lying the non-woven material and including a peripheral region, extending beyond the biopolymer layered structure that is sealable to the skin of the subject; and a port, coupled to the semi-permeable layer, that is connectable to a negative pressure source. The medical dressing of the present invention may further comprise an adhesive layer, disposed on the peripheral region, which causes adherence of the semi-permeable layer to the skin. Further still, the semi-permeable layer may be defined by a moisture-vapor transition ratio of 1 to 1000 g/24 hr-m² (grams per 24 hour per meter squared).

The present invention contemplates a system for negative-pressure treatment of a wound of a subject, the system comprising: a bioresorbable biodegradable non-woven layer comprising a plurality of bioresorbable polylactic acid fibers, forming a core support for the absorbent wound-contacting surface, wherein the fibers are oriented to provide compression resistance and maintain paths for liquid-flow and air-flow, essentially in a direction transverse to an exterior surface, and wherein the fibers are vertically lapped and have a diameter of 0.005 to 0.020 inches; a bioresorbable and biosorbable hydrophilic surface coating on a substantial number of the fibers proximate to the wound surface; a silver-based antimicrobial bioactive agent in one or more of the core and the surface coating; a semi-permeable layer defined by a moisture-vapor transition ratio of 1 to 1000 g/24 hr-m² over-lying the core and including a peripheral region, extending beyond the core, that is sealable to the skin of the subject; an adhesive layer, disposed on the peripheral region, that causes adherence of the semi-permeable layer to the skin; and a port, coupled to the semi-permeable layer, that is connectable to a negative pressure source.

The present invention contemplates a method of treating a wound, the method comprising: providing a wound dressing comprising (i) a bioresorbable biodegradable non-woven material comprising a plurality of bioresorbable fibers incorporating a bioactive agent and having a wound-contacting surface, wherein the fibers are oriented to provide compression resistance and maintain paths for liquid-flow and air-flow, predominately in a direction transverse to an exposed surface; and (ii) a bioresorbable hydrophilic surface coating on a substantial number of the fibers; applying the wound dressing to said wound with the wound-contacting surface in contact with the surface of the wound, thereby protecting the wound by providing resistance to compression and maintaining paths for air-flow and fluid-flow; and removing exudate from the wound.

The present invention further contemplates that the fibers are natural fibers selected from one or more of the group consisting of cotton, bamboo and sisal and/or one or more synthetic fibers of polymers selected from but not limited to the group comprising polylactide, polyglycolide, poly-L-lactide and poly-DL-lactide.

Further, the present invention contemplates that the bioresorbable hydrophilic surface coating is on a substantial number of the fibers proximate to the wound surface and that the coating comprises one or more of cellulose, alginate, gums, starch, ethylene glycol, polyoxethylene and polylactic acid. The present invention further contemplates that the bioresorbable and biosorbable surface coating wicks exudate from the wound. Further still, the present invention contemplates the removal of the exudate from the surface coating by a vacuum procedure (i.e., a negative pressure wound dressing) and that the exudate is removed by a vacuum procedure.

In addition, the present invention contemplates that the wound dressing has fibers wherein each of the fibers in the plurality of fibers has a diameter of 1 μm to 1 mm. The present invention contemplates that the diameter of the fibers is selected to provide a desired compression resistance between a range of 0% and 50%. The present invention also contemplates that these fibers may be processed by one or more of being cut into staple of selected length, carded, air-layered, needle-punched, vertically lapped, spirally wound or thermally bonded. The present invention further contemplates that a bioactive agent is incorporated into one or more of the fibers and the surface coating and that the bioactive agent may be antimicrobial. Yet further still, the present invention contemplates that the antimicrobial bioactive agent comprises a mixture of two or more components selected from a group consisting of (i)—silver ion-exchange particles and (ii) silver in the form of a water-soluble matrix. Yet further still, the present invention contemplates that the wound dressing of the present invention is placed into the wound so as to fill 25% or more of the volume of the wound.

The present invention additionally contemplates a method of treating a wound in a subject, the method comprising: providing a wound dressing comprising (i) a bioresorbable biodegradable non-woven material comprising a plurality of bioresorbable polylactic fibers, the core having a wound-contacting surface for contacting a surface of the wound, wherein the fibers are oriented to provide compression resistance and maintain paths, for liquid-flow and air-flow, essentially in a direction transverse to an exposed surface, and wherein the fibers are vertically lapped and have a diameter of 0.005 to 0.020 inches; (ii) a bioresorbable and biosorbable hydrophilic surface coating on a substantial number of the fibers proximate to the wound surface; and (iii) a silver-based antimicrobial bioactive agent in the core and in the surface coating; incorporating the wound dressing into the wound with the wound-contacting surface in contact with the surface of the wound, in such a matter as to fill 25% or more of the wound and provide resistance to compression and maintain paths for air-flow and fluid-flow; over-lying the core with a semi-permeable layer, defined by a moisture-vapor transition ratio of 1 to 1000 g/hm² including a peripheral region, extending beyond the core, that is sealable to the skin of the subject by an adhesive layer, disposed on the peripheral region, that causes adherence of the semi-permeable layer to the skin; and coupling the semi-permeable layer to a port that is connectable to a negative pressure source; applying negative pressure within the wound; removing exudate from the wound and/or wound dressing by, preferably, a vacuum device or procedure.

The present invention contemplates a cross-linked, biopolymer gelled composite comprising a gel-forming polymer selected from one or more of the group consisting of alginates, pectin substances and carrageenans, a water soluble plasticizer and a crosslinking polyvalent cation; wherein the weight ratio of the plasticizer to the gel-forming polymer is about 10:1 to about 2:1 and, wherein the plasticizer comprises more than 45-75 wt % of the composite and the composite is essentially homogeneous. The present invention further contemplates that the gel-forming polymer is comprised of one or more alginates. The present invention further contemplates that the water soluble plasticizer is selected from one or more of glycerin and sorbitol. The present invention further contemplates that the ratio of plasticizer to gel-forming polymer is about 8:1 to about 2:1. The present invention further contemplates that the ratio of plasticizer to gel-forming polymer is about 6:1 to about 4:1. The present invention further contemplates that the cross-linked, biopolymer gelled composite further comprises a bubble forming agent.

The present invention further contemplates that the cross-linked, biopolymer gelled composite further comprises one or more additives selected from bioactive agents, cosmetic agents, thixotropic agents, thermo-sensitive agents, and thermo-tactic agents. The present invention further contemplates that the polyvalent cation of the cross-linked, biopolymer gelled composite is selected from one or more of a group consisting of calcium ion, magnesium ions, chromium ions and zinc ions. The present invention further contemplates that the polyvalent cation may further be selected from one or more of a group consisting of multiple units of monovalent sodium ions, multiple units of monovalent potassium ions and multiple units of monovalent silver ions and multiple units of multivalent silver ions.

The present invention further contemplates that the cross-linked, biopolymer gelled composite when wet with physiological fluid maintains a neutral pH or essentially a neutral pH. The present invention further contemplates that the gel-forming polymer comprises one or more carrageenans. The present invention further contemplates that the cosmetic agent of the cross-linked biopolymer gelled composite is selected from one or more of the group consisting of natural oils, vitamins, collagens, oligoproteins, hyaluronan, hydrolysates, humectants, and UV filtering and inhibiting agents including but not limited to methyl paraben and propyl paraben. The present invention further contemplates that the natural oil of the cross-linked, biopolymer gelled composite is selected from one or more of avocado oil, coconut oil, olive oil and other generally recognized as safe (GRAS) vegetable oil or sustainable sourced oils. The present invention further contemplates that the humectants of the cross-linked, biopolymer gelled composite is selected from one or more of hyaluronans, sorbitol and glycerin.

The present invention further contemplates that the bubble forming aeration agent is one or more of hydroxy propyl methyl cellulose (HPMC), and hydroxy propyl cellulose (HPC). The present invention further contemplates that the amount of polyvalent cation in the composite is sufficient to saturate 10% to 60% of the gelling sites of the gel-forming polymer. The present invention further contemplates that the cross-linked, biopolymer gelled composite has an absorbency of at least about 10 grams of aqueous liquid per gram of gelled composite. The present invention further contemplates that the cross-linked, biopolymer gelled composite has an absorbency of up to about 100 grams of aqueous liquid per gram of gelled composite. The present invention further contemplates that the cross-linked, biopolymer gelled composite has an absorbency of about 10 to about 17 grams of aqueous liquid per gram of gelled composite.

The present invention further contemplates that the cross-linked biopolymer gelled composite is self-supporting. The present invention further contemplates that the cross-linked, biopolymer gelled composite further comprises one or more of a woven and a non-woven substrate. The present invention further contemplates that the substrate further comprises a cohesive composition. The present invention further contemplates that the cohesive composition comprises one or more of a natural rubber latex, a synthetic rubber latex, poly-isoprene, poly-chloroprene, polyurethane, poly lactic acid, poly caprolactone/polyurethane, poly caprolactone/polylactic acid, or polylactic acid/polyurethane.

The present invention further contemplates that the cross-linked, biopolymer gelled composite of the present invention further comprises an excipient containing at least one bio-active agent. The present invention further contemplates that the bioactive agents is an antimicrobial agent. The present invention further contemplates that the antimicrobial is selected from one or more of silver, silver salts, zeolites containing one or more of silver and copper, copper, copper salts, chlorohexidine, quaternary ammonium salts, iodine and chitosan. The present invention further contemplates that the excipient functions as a Controlled Sustained Release (CSR) delivery system and, wherein one or more of the bio-agents are selected from a group consisting of anti-inflammatory agents, collagen, antibacterial and antifungal agents, antibiotics, antiseptics, cancer therapeutics, natural and synthetic nitric oxide generating materials, synthetic nitric oxide stimulating materials, fibroblast and epithelial cell chemotactic hyaluronans and humectants.

The present invention further contemplates that the gelled composite of the cross-linked, biopolymer gelled composite is cast on both sides of the same woven or non-woven substrate. The present invention further contemplates that the woven and non-woven fibers comprise one of more of PLA, SMS-PP, reticulated PUR, foams and Alginate.

The present invention further contemplates that the cross-linked, biopolymer gelled composite of the present invention further comprises an absorbent thermal sensitive material. The present invention further contemplates that the absorbent thermal sensitive material is selected from a group consisting of poly (N-isopropyl acrylamide), poly (Vinyl Lactam), hydroxypropyl cellulose, and methyl cellulose, wherein said thermo-sensitive absorbent material at ambient temperature, will release its moisture in a controlled and sustained manner upon reaching body temperature at the point of contact. The present invention further contemplates that the cross-linked, biopolymer gelled composite of the present invention further comprises a structural foam substrate that resists compression when used with Negative Pressure Wound Therapy.

### BRIEF DESCRIPTION OF DRAWINGS

The foregoing features of the invention will be more readily understood by reference to the following detailed description, taken with reference to the following accompanying drawings, in which:

FIG. 1 represents a perspective view of a system for the delivery of a biopolymer gel-forming fluid as described by the invention.

FIG. 2 shows a model of a layered structure of a cross-linked biopolymer gelled dressing (18) cast onto a release carrier and removed for use. The biopolymer gel is coated with a film (17) containing active ingredients to be delivered onto the wound.

FIG. 3 depicts a model of a layered structure of a cross-linked biopolymer gelled dressing (18), coated with a film (17) containing active ingredients to be delivered into the wound. The biopolymer gelled layer (18) is cast first onto a breathable, barrier substrate (19).

FIG. 4 describes a model of a layered structure of a cross-linked biopolymer gel dressing or wound packing material comprised of a biocompatible, breathable, core (19) coated on both sides with a cross-linked biopolymer gel (18) which is coated with a film (17) containing bio-active ingredients to be delivered to the wound interface.

FIG. 5 shows a model of a layered structure of a cross-linked biopolymer gel (18) coated with a film (17) containing bio-active ingredients supported by an assemblage (20) of non-woven fibers such as PLA and/or Alginate and/or woven or laid cotton fibers.

FIG. 6 defines a model of a layered structure of a cross-linked biopolymer gel (18) coated with an optional film (17) containing bio-active ingredients cast onto an assemblage (20) of woven or non-woven fibers supported by a biocompatible, breathable, barrier substrate (19).

FIG. 7 shows schematic of a generic meltblown fiber manufacturing line.

FIG. 8 shows schematic of non-woven calendering.

FIG. 9 shows experimental trial matrix and performance data for different PLA fiber diameters.

FIG. 10 shows magnified photograph of PLA fibers from 0.015 inch nozzle.

FIG. 11 shows polylactic acid (PLA) non-woven in a cross-section of the layer with fiber direction being transverse to an exterior surface.

FIG. 12 shows additionally magnified, PLA non-woven in a cross-section of the layer with fiber direction being transverse to an exterior surface.

FIG. 13 shows additionally magnified, PLA non-woven in a cross-section of the layer with fiber direction being transverse to an exterior surface.

FIG. 14 shows a top and bottom one zone convection heating apparatus for bench-top drying process development.

FIG. 15 defines a model of a layered structure of a cross-linked biopolymer gel composite (18) coated with an optional film (17) containing bio-active ingredients cast onto a mini-log of cohesive elastic bandage (22) whose width is ≧55″ with an unstretched length of 1.1 linear yard, for example.

FIG. 16 illustrates a model of a layered individual bandage roll converted from the mini-log of cohesive elastic bandage (22) depicted in FIG. 15, comprising a highly absorbent, cross-linked, biopolymer gel composite (18) coated with an optional film (17) containing bio-active ingredients. This converted roll can be slit and rewound to widths of 1″, 2″, 2.5″, 3″, 4″ 6″, or 12″ and/or customized to fit any size wound or body part. The length is standard at 1.1 linear yards unstretched but can be custom made to accommodate any wound, body part or application.

FIG. 17 shows a magnified high-resolution photograph of wet gel cast material of the present invention as compared to wet foam material.

FIG. 18 shows a magnified high-resolution photograph of the cross-sectional area of the wet gel cast material of the present invention as compared to the cross-sectional area of the wet foam material.

FIG. 19 shows active layer deposition on negative pressure wound therapy foam.

FIG. 20 shows Active layer deposition on negative pressure wound therapy foam without occluding cells.

### DETAILED DESCRIPTION OF THE INVENTION

In the specification, examples and claims unless otherwise indicated, percent is defined as “percent by weight”. Except where indicated by context, terms such as “gel forming biopolymer,” “gel forming polymer,” “gelling agent,” “pH modifier,” “aeration or bubble forming aid (agent),” water soluble plasticizer,” “divalent cations,” and similar terms, also refer to mixtures of said materials. All temperatures are recorded in ° C. (Celsius) unless otherwise indicated.

As used herein, the term “alginate” refers to salts of alginic acid and modified alginates. Alginic acid, which is isolated from seaweed, is a polyuronic acid made up of two uronic acids: D-mannuronic acid and L-guluronic acid. The ratio of mannuronic acid and guluronic acid varies with factors such as seaweed species, plant age and part of the seaweed (e.g., stem, leaf). Alginic acid is substantially insoluble in water. It forms water-soluble salts with alkali metals, such as sodium, potassium, lithium, magnesium, ammonium and the substituted ammonium cations derived from lower amines, such as methyl amine, ethanol amine, diethanol amine, and triethanol amine. The salts are soluble in aqueous media above pH 4, but are converted to alginic acid when the pH is lowered below about pH 4. A thermo-irreversible water-insoluble alginate gel is formed in the presence of gel-forming ions (polyvalent cations, as are known to one of ordinary skill in the art), e.g. calcium, magnesium, chromium, barium, strontium, zinc, copper (+2), aluminum, and mixtures thereof, at appropriate concentrations. The alginate gels can be solubilized by soaking in a solution of soluble cations or chelating agents for the gel-forming ions, for example EDTA, citrate and the like. In the instance of calcium ions, calcium chloride is used most often. Calcium alginate gel is formed when the calcium ions, in the calcium chloride, react with the alginate or alginate containing mix, as the calcium ion diffuse into the mix containing alginate.

As used herein “hyaluronic acid” refers to hyaluronic acid (HA), salts thereof and modified hyaluronates. Sodium hyaluronate is an abundant glycosaminoglycan found in the extracellular matrix of skin, joints, and eyes as well as most organs and tissues of all higher animals. Non-animal derived HA may be fermented from Streptococcus zooepidemicus. Hyaluronic acid from a non-animal source is preferred for use in the present invention. Hyaluronic acid is a linear copolymer composed of (β-1,4)-linked D-glucuronate (D) and (β-1,3)-N-acetyl-D-glucosamine (N). The coiled structure of hyaluronate can trap approximately 1000 times its weight in water. These characteristics give the molecule advantageous physicochemical properties as well as distinct biological functions and is desirable for use as a building block for biocompatible and biointeractive materials in pharmaceutical delivery, tissue engineering and visco-supplementation.

Hyaluronic acid or hyaluronate is a natural component in mammalian organisms and is enzymatically biodegradable by hyaluronidases. The half-life of hyaluronate in endothelial tissue is less than a day, and the natural turnover of the polymer in adults is approximately 7 g a day. As is known to one of ordinary skill in the art, a mild to moderate covalent modification of hyaluronan will increase the in vivo stability and retention time from days up to months or a year.

Hyaluronic acid is thought to play an important role in the early stages of connective tissue healing and scarless fetal wound healing and regulates cell mobility, adhesion and proliferation and is especially useful in tissue engineering and tissue regeneration applications. HA is known to be chemotactic with respect to fibroblasts and epithelial cells. The presence of hyaluronans in the wound bed attracts said fibroblasts and epithelial cells to the wound site, initiating granulation and re-epithelialization of the wound. In addition to the role of hyaluronans as bioactive wound healing agents, they are also defined and utilized as cosmetic agents with respect to their humectant properties, as described in the Summary of Invention herein.

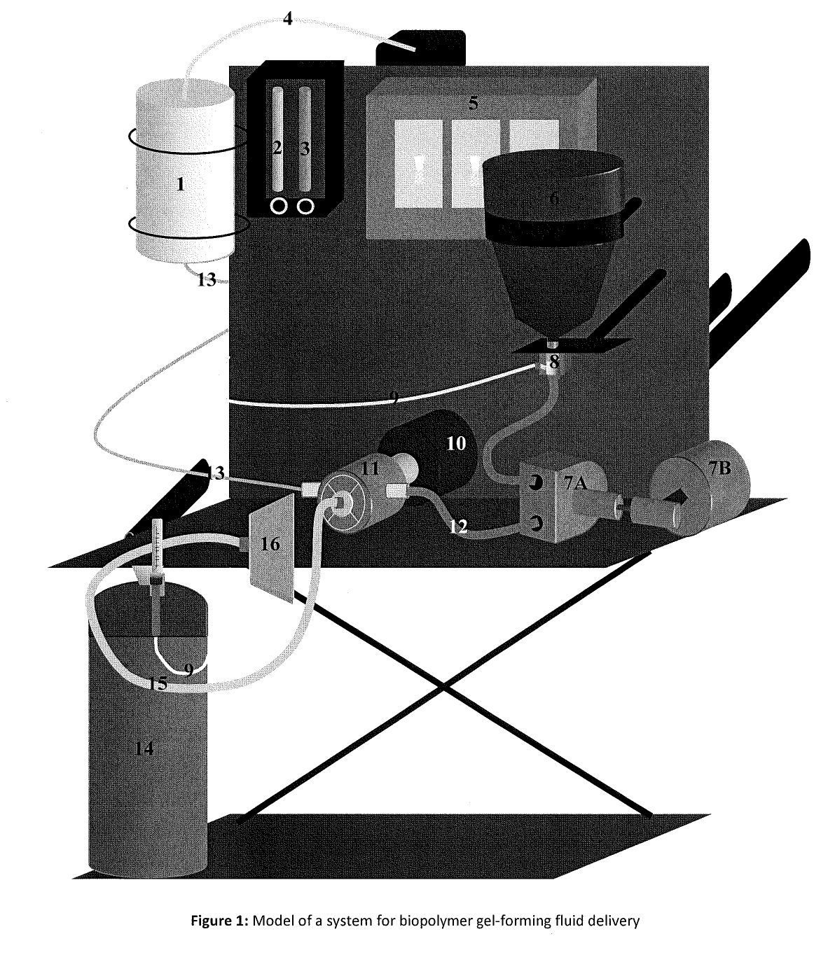

As shown in FIG. 1, the biopolymer component is introduced into the biopolymer hopper (6) and the pH modifying component is dispensed into the pressure pot (1) which is connected to compressed air by tubing (4). The compressed air is set between 0-60 psi and more favorably between 45-55 psi. The machine is engaged by first switching on the mixer motor (10) and the peristaltic pump (7A) motor (7B) with switches located on the motor switch board (5). The Nitrogen (N₂) flow rate through the N₂ line (9) and into the injection port (8) is maintained between 400-800 mL/min and preferably between 500-700 mL/min. The respective solutions from the biopolymer hopper (6) and the pressure pot (1) are independently introduced into the mixer (11). The residence time of the solutions in the mixer corresponds to the flow rate of the biopolymer solution. The pH modifying solution is introduced directly into the mixer (11) at a flow rate of 20-30 mL/min. The blended biopolymer solution is then pumped through the die head (16) and cast onto the substrate.

In one aspect, the invention describes the formation of a cross-linked biopolymer gelled composite where the biopolymer component is comprised of an aqueous dispersion of a gel-forming biopolymer, a water soluble plasticizer, a gelling agent and a bubble forming aid (agent). The composite may also comprise a pH modifying component that comprises an aqueous solution of a weak acid, with or without a water soluble plasticizer. The gel-forming biopolymer may be selected from alginates, glycol alginates, pectins, carrageenans and mixtures thereof. A preferred gel-forming polymer is alginate and makes up from 1% to 10% of the biopolymer component. As the molecular weight of the alginate increases, so does the wet and dry mechanical strength of the resulting biopolymer gelled composite. A moderately high molecular weight between 100 KD and 300 KD affords excellent structural integrity when wet while not exceeding the viscosity requirements of the process. A preferred gelling agent is calcium carbonate, which not only provides the cations necessary for gel formation, but it also provides a buffering effect and can produce a biopolymer gelled composite which maintains a neutral pH upon contact with physiological fluids. The concentration of the gelling agent affords a means to control the cross-link density of the gelled composite which allows for the design of specific physical and mechanical properties expressed by the gelled composite. Also, any available gelling sites in the final product can be utilized to bind monovalent cations such as silver, sodium and potassium, which may serve as preservative, anti-septic or a general anti-microbial within the composite itself. The preferred water soluble plasticizer is defined as sorbitol and/or glycerin and will impart softness and flexibility (pliability) to the final product. Although the plasticizer typically comprises about 50 wt % of the cross-linked biopolymer gelled composite, it is notable that as the amount of plasticizer in the formulation increases, the absorbency of the gelled composite decreases. Polymeric bubble forming aid (agent) such as the surface active hydrocolloids including but not limited to hydroxy propyl methyl cellulose (HPMC), hydroxypropyl cellulose (HPC), and methyl cellulose (MC) can be utilized to create small bubbles which remain intact as gelation occurs and are substantially non-leachable. Methyl cellulose or hydroxypropyl cellulose may be preferred for wound dressing applications where it is desirable to absorb and hold moisture in the dressing under ambient conditions and then releasing the moisture to the wound site upon contact with the skin, as would be necessary for burn injury wounds. Optionally, surfactants such as the non-ionic ethoxylates of sorbitan esters can be used in concert with the polymeric bubble forming agent for more refined control over bubble size and longevity. Absorbency of the gel (gelled) composite is at least 10 grams of aqueous liquid per gram of gelled composite, about 10 to 17 grams of aqueous liquid per gram of gelled composite, up to about 100 grams of aqueous liquid per gram of gelled composite.

The pH modifying component is comprised of an aqueous solution of a weak acid such as glucono delta lactone (GDL), which slowly reduces the pH allowing gelation to occur in a very controlled manner, affording formation of a mechanically homogeneous composite with optimum strength. Optionally, a water soluble plasticizer can be added to the pH modifying solution for increased softness and pliability. In addition, the density, absorbency and softness of the gelled composite can be adjusted by varying the blending time with longer times affording lighter, fluffier and softer composite materials.

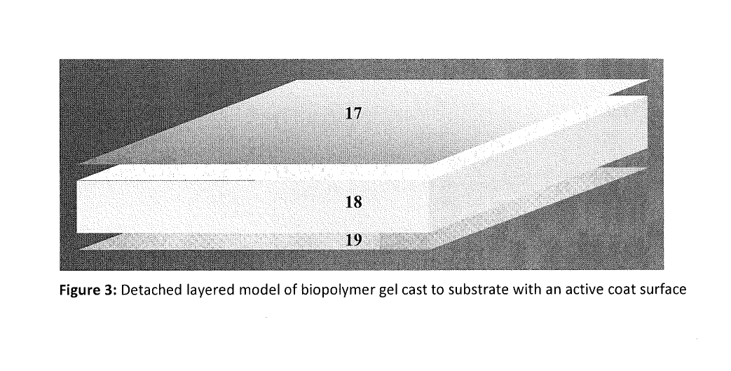

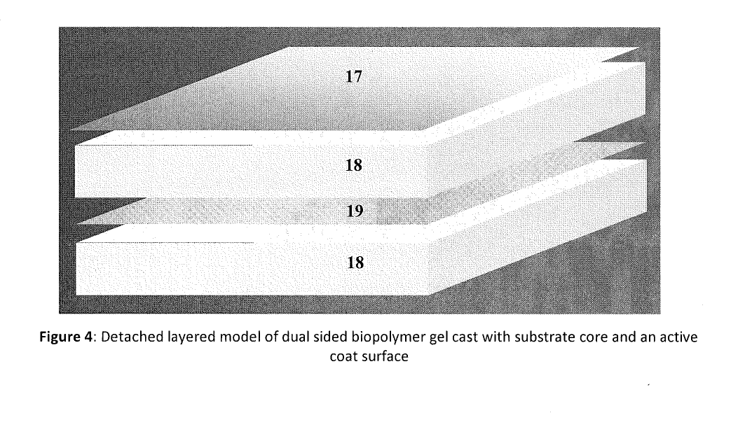

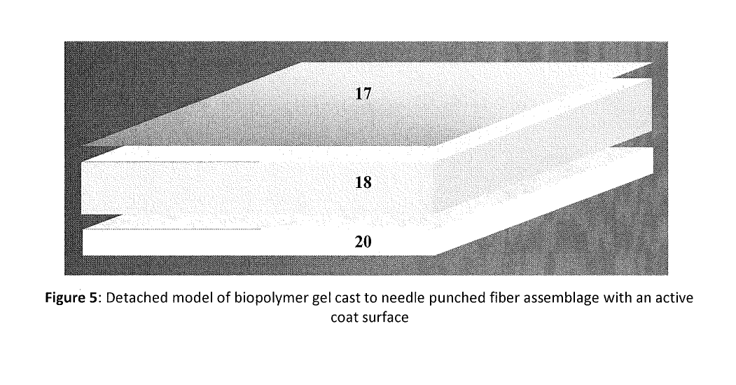

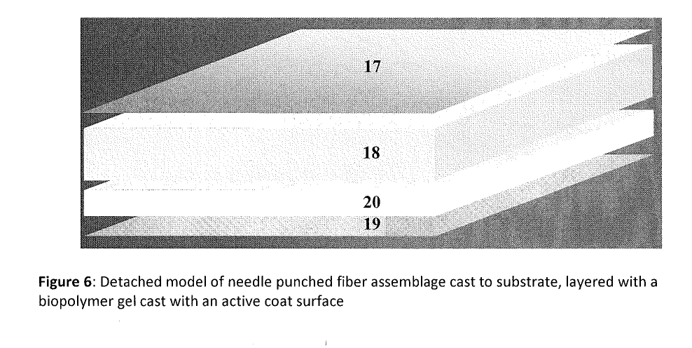

The wet gelled composite may be cast as a layer or as a shaped article. For example, the gel may be cast as a layer on a substrate, which may be a woven material such as a cohesive elastic bandage used for the treatment of wounds requiring compression therapy or non-woven fibrous article, a film, or another cross-linked, biopolymer gelled composite (FIG. 3). The substrate may comprise, for example, an assemblage of fibers or yarns, such as cotton, linen, silk, nylon, polyester, rayon, polysaccharide such as alginate, polylactide and blends thereof (FIG. 5 & FIG. 6), a non-woven material, such as TYVEK® spun-bond polyethylene, or a material such as paper or a polymer film. Two or more layers of cross-linked, biopolymer gel with the same and/or different physical properties and/or chemical ingredients (such as different active ingredients, colors, etc.) can be laminated together to create multiple layered composites (i.e., a layered structure) with various benefits, such as the delivery of otherwise non-compatible beneficial agents, at the same or different times. This technique can be used to build in desired release characteristics of beneficial agents, desired texture, absorbency profiles and desired appearance. This can be performed by incorporating two or more layers of dry sheets of gel composite. Alternatively, a second layer of wet gel can be cast onto the substrate of the first layer thus the original substrate becomes a core material supporting a layer of gelled composite on either side (FIG. 4). This double sided composite can not only function similarly to the multilayered embodiment (layer structure) described above, building in uniquely desired release characteristics, textures, absorbency profiles, etc., but can serve as highly absorbing, soft, pliable, non-fraying, wound packing material that may also function as a controlled and sustained release delivery system. The gelled composite may be cast as a thin composite layer with a thickness of up to about 1 mm. In addition, the gelled composite may be cast as a thick composite layer having a thickness from about 1 mm to about 30 mm. A convenient dry thickness for a wound dressing is about 2 mm to about 10 mm, typically about 5 mm. Further, the gel composite can be self-supporting, which means it does not need any other “carrier” or supporting laminate, layer, structure. It would therefore have sufficient strength and integrity to be a material layer on its own. Further still, the gel composite can be deposited, cast, or layered onto other materials and layers as described herein.

The cross-linked, biopolymer gelled composite is useful as a wound dressing. The wound dressing combines many of the desirable wound dressing properties, including, for example, high absorbency, high flexibility, vertical wicking, non-adherence to the wound, high dry strength, high wet strength, calcium donation and a non-shedding matrix. Further, antimicrobial agents, such as silver, silver salts and/or chitosan, etc., may be incorporated into the dressing.

Wound dressings are the primary dressing placed in direct contact with a wound or as near as practical against the wound. Wound dressings may be used on injured tissue and for bodily fluid drainages where control and management of fluid and secretions is desired. The dressings may, if required, be secured into position with any suitable secondary wound dressing such as a wrap, tape, gauze or pad. Wound dressings are temporary, however, and are not incorporated into the healing tissues. For wound dressing applications, the gel typically will maintain a neutral pH (approximately a pH of 6.5 to 7.5 or 6.8 to 7.2) upon contact with physiological fluids.

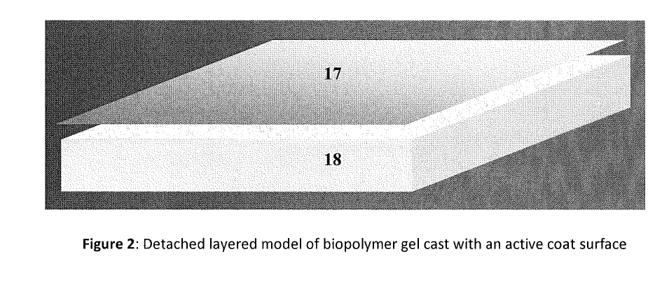

The wound dressing is, in one embodiment, contemplated to be a layered structure and may additionally comprise a layer of the gel on a substrate. The substrate may be a woven or non-woven fibrous article, a film or other cross-linked, biopolymer composite. Alternatively, the cross-linked, biopolymer gelled composite may be used as a wound dressing without a support (see FIG. 2). The dressing may also contain a wicking layer between the gelled composite and the substrate. The wicking layer not only provides absorbency but, more importantly, it facilitates moisture to move from the wound facing side of the dressing to the back of the dressing where it escapes out of the dressing through a breathable backing. It should have good wicking properties so that moisture can be spread over as large a surface area as possible, thus increasing evaporation. The overall effect of this layer is to draw moisture from the gelled composite, thus decreasing the chances of wound maceration and to increase evaporation through the backing of the dressing. The wicking layer may be formed of several plies (which may or may not be the same) if desired, but it is preferred that the total thickness of the wicking layer does not exceed about 1 mm to 5 mm. Suitable materials for the wicking layer include nonwoven, woven and knitted fabrics. Nonwoven viscose fabrics such as those conventionally used for making nonwoven surgical swabs are preferred, but many alternative fabrics, particularly other cellulosic fabrics, or hydrophilic biopolymers such as modified PLA could be used in their place.

The cross-linked, biopolymer gelled composite and/or the excipient active coating on its surface may be used as a controlled release delivery system, or as a delivery system for beneficial agents such as, for example: collagen, antibiotics, antibacterial agents, antifungal agents, antiseptics, anti-inflammatory agents, agents for the treatment of cancer, nutritional agents, living cells, etc. The hydrated gelled composite layer presents a low diffusion barrier to water soluble molecules so that water soluble beneficial agents will rapidly diffuse out of the hydrated composite. The delivery system may be used directly or the delivery system may be pre-hydrated in water or an aqueous liquid, such as physiological saline.

For all of the advances which have been made in treating chronic and moderately to highly exudating wounds, there is still a need for advanced wound dressings for burn injury wounds. This invention utilizes the cross-linked biopolymer gelled composite in all of its embodiments detailed herein with the incorporation of poly (N-isopropyl acrylamide) (PNIPAAM) along with an active coating on the gel surface, as previously described, to deliver healing ingredients to the burn injury wound. At ambient temperatures≦34° C., poly (N-isopropyl acrylamide) is highly hydrophilic and highly absorbing of wound exudate which it tightly holds. As the temperature rises to a minimum of just below body temperature and especially at the elevated temperature of the burn wound, the PNIPAAM becomes hydrophobic and releases active medicinal or cosmetic agents which it absorbed at lower temperatures. Alternatively, or in combination with PNIPAAM, an embodiment could utilize one or more of poly (vinylcaprolactam) and methyl cellulose which also are highly hydrophilic≦34° C. and hydrophobic at or above body temperature. This technology will be a significant improvement upon the existing burn therapy dressings, including the much over rated, but best available, hydrogel burn dressings.

Nitric oxide is a high potential wound therapy due to its considerable antimicrobial activity and its ability to induce angiogenesis and re-epithelialization. The topical use of NO resulted in the acceleration of the wound healing process in murine models, while the use of NO inhibitors, topically or systemically, has increased the healing time. Several studies have been performed using NO donors in colloids and evidenced the beneficial effect of NO in the granulation and closing of the wounds for Diabetic foot ulcers (DFU) in animal models. However, these therapeutic alternatives are limited by the short half-life of the nitric oxide produced and the failure of the devices available to guarantee a sustained release of NO to the affected area.

This invention contemplates the incorporation of the NO-releasing poly (acrylonitrile)-based materials or, alternatively, the natural product pycnogenol into or onto the surface of biocompatible dressings comprised of a cross-linked biopolymer gelled composite to accelerate wound closure, alleviate pain and reduce the cost of healing recalcitrant wounds. To date, the only vehicles which have been prepared to deliver NO at the wound site are topical applications of creams, gels and emulsions. Although the release of nitric oxide from these topical ointments was not optimal for dosing and duration, they did show some minor healing improvements vs. treatment without nitric oxide (NO), which is somewhat encouraging. A need therefore exists to provide a product suitable for use, for example, in wound management and this invention is directed to this need and the other yet to be satisfied needs described herein.

Venus leg ulcers are an example of a recalcitrant wound. Venus leg ulcers, relatively common in older people and in diabetic patients, become infected easily. Occasionally, a persistent venous ulcer can present with development of skin cancer around the edge. Leg ulceration is a major problem affecting about 2% of the population at some point during their lives. Although many of those affected are part of the senior population, about one third of leg ulcer patients present before the age of 50 and two thirds present before the age of 65.

Leg ulcers can result in a high death rate and a significant financial burden. Treatment often extends over a long period of time and, depending upon the degree of progression, can be very costly.

The role of the venous system is to return blood to the heart. The venous system of the legs has deep veins as well as superficial and communicator veins. The veins have valves which act as a shunting system to allow blood to flow back to the heart. Contraction of the calf muscles assists the shunting system against gravity. Venous ulcers form when the blood flow through the legs is reduced causing the blood to pool in the leg veins. Then, the pressure increases in the veins and the capillaries (the tiny blood vessels that connect the arteries and the veins). The increased pressure of blood in the leg veins is due to blood pooling in the smaller veins next to the skin.

The blood tends to pool because the valves in the larger veins are damaged. The valves may be damaged by a previous thrombosis (blood clot) in the vein or due to varicose veins. Gravity causes blood to flow backward through the damaged valves and pool in the lower veins. When the muscles of the leg are weakened, they can no longer create the required pressure during contraction to force the blood up through the veins into the inferior vena cava and eventually to the heart. As a treatment for the early stages of damaged lower leg veins or varicose veins and as a preventative (or treatment) of venous leg ulcers, compression stockings or compression bandages are used to apply a graduated pressure to the leg (higher at the ankle and lower at the upper calf) to support the blood flow from the lower leg veins back to the heart.

Although compression stockings, which are manufactured to order, are useful in the support of varicose veins and the prevention of some venous leg ulcers, they are not useful in the treatment of many advanced leg ulcers because they cannot accommodate the required wound dressings. In conjunction with a wound dressing in the treatment of leg ulcers, only compression bandages are indicated as effective treatment. A need therefore exists to provide a product suitable for use in the treatment of painful, costly, recalcitrant wounds such as, but not limited to, venous leg ulcers.

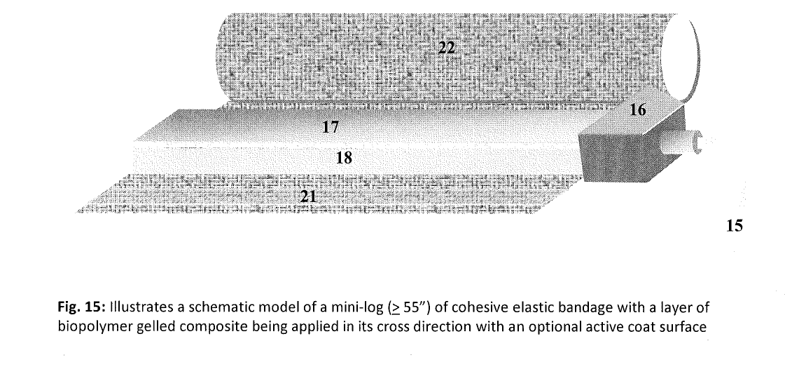

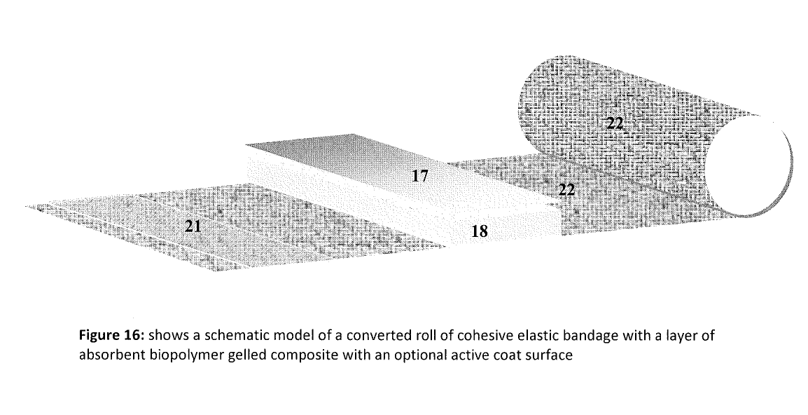

Another aspect of this invention therefore is a compression wound dressing with a built in Controlled Release System to deliver healing medicaments, such as nitric oxide, directly to the wound to aid in the treatment of venous leg ulcers and other chronic, slow healing wounds (see FIG. 15). This embodiment can be defined by a substrate such as a cohesive elastic bandage of a type such as Cohere (a registered trademark of Tape-O Corp of Dover, N.H.) or Coban (a registered trademark of the 3M Company, of Minneapolis, Minn.), which supports a cross-linked biopolymer gelled composite layer (see FIG. 16). As previously described herein, the biopolymer gelled composite layer with or without the optional active coating, provides a controlled and sustained release of bioactive ingredients into the wound. The biopolymer gelled composite is absorbent, breathable, conformable and comfortable. The cohesive elastic bandage substrate imparts the ability to apply controlled compression to the wound. The combination of absorbency and compression facilitates hemostasis and as such may become the dressing of choice for EMT and other in-field practitioners to manage actively bleeding wounds.

The cohesive composition may include but is not limited to at least one of natural rubber latex and/or a latex-free cohesive such as a synthetic rubber latex, poly-isoprene, poly-chloroprene, polyurethane, poly lactic acid, poly caprolactone/polyurethane, poly caprolactone/polylactic acid, or polylactic acid/polyurethane.

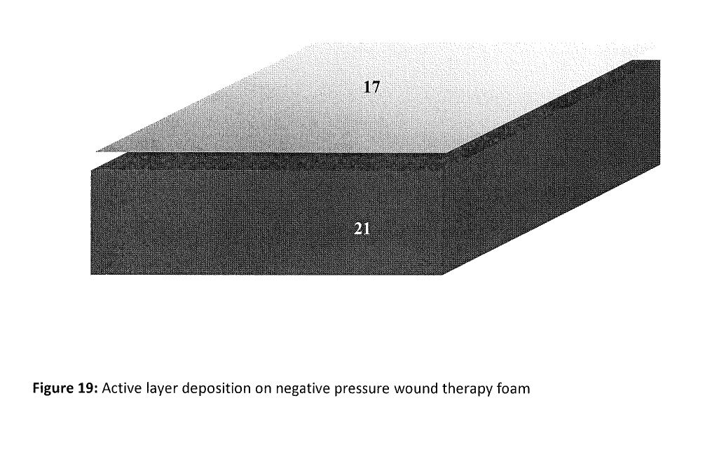

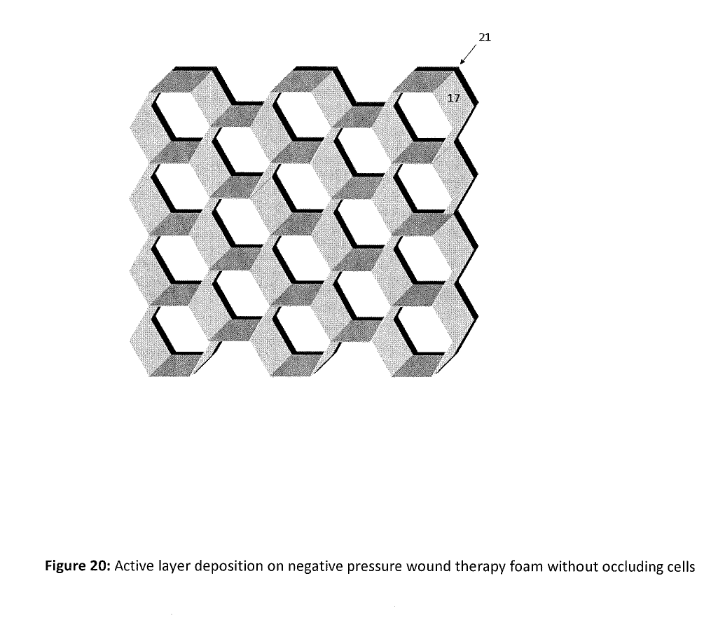

An additional embodiment of the present invention is in combination with a standard negative pressure wound therapy (NPWT) foam component such as KCl GranuFoam™ (San Antonio, Tex.), as shown in FIG. 19; wherein the GranuFoam™ type dressing is the substrate for a layer of the cross-linked biopolymer gelled composite coating. This coating imparts the controlled and sustained delivery of the aforementioned actives such as antimicrobials, anti-infectives, collagen, hyaluronans, and nitric oxide for enhanced or accelerated healing of recalcitrant wounds and/or anti-inflammatory and analgesic agents. Alternatively, FIG. 20 illustrates the selective coating of said cross-linked biopolymer gelled composite on the interior and exterior surfaces of the cellular structure of the NPWT foam dressing without occlusion of the cells. In addition to providing the controlled and sustained release of enhanced wound healing actives, the coating of this invention affords a non-stick surface for easy, pain-free removal from the wound.

As used herein, the term “polymer” refers to thermoplastic, natural, naturally-derived, synthetic, biopolymers and oligomers, as well as mixtures, thereof. As used herein, the term “oligomer” refers to a low molecular weight polymer of two or more repeating monomeric units. Polymers specifically include, but are not limited to, Polylactic Acid (PLA); PolyCaproLactone (PCL) and PolyHydroxyAlkanoate (PHA) alone or in blends/alloys or as copolymers.

The non-woven material layer, i.e., a layered structure comprising one or more layers), prepared according to embodiments of the invention described herein utilizes natural or naturally-derived fibers, especially poly (lactic) acid, as the basis of the “backbone”, non-collapsible wound dressing support structure. Its breathable characteristics provide both moisture management and protection from undesirable substances such as bacteria, viruses and other exogenous contamination as from fluids. The medical dressing of the present invention is particularly well adapted to uses of low to medium wound exudate, uses under negative pressure, is non-adherent, has the ability to deliver antimicrobial agents to the wound site, and has inherently low bioburden. The non-woven material is completely biodegradable; its composition can be varied to provide the ability to control the degradation. The non-woven layer can also be modified with hydrophilic and hydrophobic materials to vary its ability to hold or absorb moisture in the wound bed or for cross-linking properties with the other layers in the medical dressing. The construction of the non-woven material layer and the dressing is such that it presents no sharp edges. The density of the non-woven layer and also of the dressing may be varied as well. Furthermore, the non-woven material on which the majority of the dressing structure rests can fit easily into the irregularly shaped wound bed by cutting and folding sheets of three-dimensional scaffolds or by holding the wound dressing at the wound with a secondary dressing.

More specifically, in some embodiments of the invention of the non-woven layer, the non-woven materials have a fibrous structure as described herein.

In one embodiment of the invention, the non-woven material includes a bioresorbable layer having a plurality of bioresorbable fibers and a bioresorbable hydrophilic surface coating on a substantial number of the fibers if so desired. The layer has a surface for cross-linking or “engaging” with the other layers of the medical dressing structure. The fibers are oriented to provide compression resistance (also referred to as % compression set) and maintain paths, for liquid-flow and air-flow, preferentially in a direction transverse to an exterior surface. The percent compression set is a measure of the permanent deformation of a material after it has been compressed between two metal plates for a controlled time period and temperature condition. The standard conditions are 22 hours at 70° C. (158° F.). The subject material is compressed to a thickness given as a percentage of its original thickness, usually 50%. Compression set is expressed as the percentage of its original thickness that remained “set”. For example: If a 2″×2″×1″ sample measured 1.00 inch before compression and 0.95 inch after the test, it is reported to have a compression set value of 5%, i.e., it did not recover 5% of its original thickness. When used with an NPWT device, the fibrous non-woven layer provides resistance to compression under vacuum. This is critical as the applied vacuum or negative pressure must penetrate the wound bed to be functional. The orientation of the fibers within the layer can be arranged such that they provide resistance to this crushing effect and maintain transverse paths for the air-flow and fluid-flow.

In some embodiments, the materials according to this invention provide the physical function of reticulated conventional foam while providing important additional features and advantages as mentioned herein.

The non-woven layer also offers bio-compatibility within the wound cavity and degrades naturally when residing in the open wound or healed wound. In some embodiments, the fibers can be in an intimate blend or arranged in layers. In some embodiments, a continuous filament nonwoven process, such as melt-blowing or spun-bond fibers, is generally used to arrange the fibers. In some other embodiments, woven fibers using the techniques of knitting and weaving can also be used. In the case of woven or knit fibers, the composite structure can provide a function similar to that of gauze. In some other embodiments, a hybrid process known as stitch-bonding can also be employed. The selection of the fabrication method and physical properties of the fibrous structure is dependent on the physical demands of the final application, from soft and flexible to rigid and non-compressible.

Examples of useful fibers are those of plant, animal, and synthetic origin, as well as fibers classified as naturally-derived origin. Examples of plant-origin fibers include, but are not limited to, cotton, bamboo, jute, flax, ramie, sisal, hemp, polyethylene blend with hybrid plant origin polymer and polypropylene blend with plant-origin polymers. Examples of animal-origin fibers include, but are not limited to, proteins such as collagen, silk and keratin. Examples of synthetic fibers include, but are not limited to, polyesters, including materials that traditionally are not found in fibrous form such as polyurethane and silicone or silicone-based fibers. In some embodiments, the preferred polymer is poly (lactic) acid (PLA) and copolymers of PLA which are biodegradable and support low bioburden.

Such biodegradable and low bioburden fibers include those based on poly (lactic) acid, also known as polylactide, and its various L, D, DL and meso configurations, including mixed L, D, and meso compositions, their various crystallinities, molecular weights, and various co-polymers. In this work, poly (lactic) acid is understood to be synonymous with poly (lactide) and both terms encompass all of the light rotating configurations of the polymer. Other synthetic fibers useful in the present invention include, but are not limited to, polyglycolide and polycaprolactone.

PLA is also bio-resorbable. The term bioresorbable refers to materials that can be broken down by the body should it not be manually removed there from. An example of such a material is a bioresorbable suture based on a poly (lactic) acid copolymer.

In our current invention, although we can utilize synthetic fibers such as polypropylene and polyethylene (e.g., polyethylene terephthalate), or paper such as recycled paper, we preferentially employ natural plant-based materials, such as natural polymers or naturally-derived meltblown nonwoven polymer fibers or filaments. One example is poly (lactic) acid (PLA), as defined above. The PLA non-woven is degradable and renewable, and has a low bioburden as opposed to, for example, recycled wood pulp. From an end-use standpoint and a processing and manufacturing standpoint, the low bioburden profile achieved with the nonwoven process precludes any heat drying that is required to destroy microbes present in a wood or tissue-based product; allowing a “cleaner” and safer system when compared to traditional alternatives such as wood pulp (e.g., paper-based products).

Another differentiating feature of PLA is that it is completely compostable, resorbable and safe in terms of cytotoxity, versus recycled pulp or synthetic fibers. One of the degradation products of poly (lactic) acid is lactic acid, which is produced abundantly in the human body.

In some embodiments, 100% PLA polymer may be used. In some other embodiments, co-polymers of PLA with masterbatch additives and/or plasticizers may be used with distinct advantages. As an example, when polycaprolactone, a degradable polymer often used in medical implants, is incorporated at up to 50% of the blend with PLA, the fibers exhibits flexibility and softness to counteract the inherent brittle nature of the PLA. Other additives such as plasticizers and lubricants aid in the fiber-spinning process.

NatureWorks (Minnetonka, Minn.) produces several grades of PLA in pellet form that can be melt processed into film or fibers and are useful in this invention. Many grades are useful however grade 6202D as a high melt-point version with the optional use of grade 6251D as a low-melt binder fiber have proven to process well in the present invention. Perstorp (Toledo, Ohio) produces PCL and, although several grades are suitable for use in the present invention, grade Capa 6800 processes well. Mirel PHA from Meta bolix (Cambridge, Mass.) is also compatible with the present invention.

When processing PLA, to maintain maximum chain length, it is important to dry the polymer in a commercial desiccant dryer such as a Conair (Cranberry Township, Pa.) “W” series machine to a moisture level below 200 ppm (parts per million). This is critical as PLA polymer is extremely hydroscopic and will acquire moisture from the air rapidly. This moisture hydrolytically degrades the polymer chains resulting in a reduced viscosity and thus product strength. If moisture levels are too high, the additional problem of steam generation and uncontrolled pressures within the extrusion system are observed.

For a production exemplification, a Davis-Standard (Pawcatuck, Conn.) single screw 30:1 2.5″ extruder (or equivalent) with melt temperatures of 350 to 425° F. and pressures of 500 to 2000 psi are achieved at the outlet. The polymer passes thru filtration to remove particulate debris and enters a pressure control zone achieved via a positive displacement Zenith (Monroe, N.C.) gear pump. Molten pressurized polymer is delivered to a melt-spinning die produced by BIAX (Greenville, Wis.). Several arrangements of nozzles, diameters, and total nozzle count can be varied to suit the polymer and final production needs. A typical spinning die contains 4000-8000 nozzles/meter of width with an internal diameter of 0.25-0.50 mm may be utilized efficiently. It must be noted that melt spinning dies produced by other suppliers such as Hills (W. Melbourne, Fla.) or Reifenhauser (Danvers, Mass.) may be used.

Heated and high velocity air is introduced into the die and both polymer and air steams are released in close proximity allowing the air to attenuate the polymer streams as they exit the die. Air temperatures of about 230-290° C. with pressures at the die at about 0.6 to about 4.0 atmospheres may be used. Following extrusion and attenuation, cool and/or moist air may be used to quench the fibers rapidly. At this point, liquids or mists can be applied to coat the surface. Surfactants, antimicrobials, or adhesives can be beneficially adhered to the fibers.

The fibers may be collected on a single belt or drum or a multiple belt or drum collector. Air is drawn from below the belt(s) or drum(s) and fibers collect in a web or matt on the surface. There are many adjustments in the entire system, temperatures, pressures, quench conditions, extrusion air velocity, suction air velocity, etc. Utilizing these process parameters, a matt can be designed to be, for example, stiff and thin or flexible and fluffy as well as producing various structures in between. For this invention, a low-density structure with fine-diameter fibers is beneficial although one of skill in the art will realize that other densities and diameters are suitable for use within the present invention. The lower density improves fluid acquisition and the small diameter maximizes surface area, which are important for the release of “actives” from the fibers.

Fiber diameters can range from approximately 1 to 1000 microns (μm) however it is possible to produce nano or sub-micron fibers via increased hot air attenuation and/or low polymer throughputs. The cost of production increases however as the overall surface area of the fibers increases. Likewise, larger fibers are easily produced when attenuation air is reduced or eliminated and/or melt pressures are increased. A compromise of cost and performance is seen in, approximately, the 5-25 micron range. Within the large number of consecutive fibers being spun, it can be important to allow a range of diameters as this has been observed to increase the loft or thickness of the structure and this provides for improved shock absorbing and cushioning properties. Different diameters can be achieved by adjusting the internal nozzle diameters and/or air velocity at specified nozzles or by directing external cooling air toward certain fiber streams.

The fibers can be formed in a continuous melt spinning operation and arranged into a web as described above. The fibers can also be cut into staple and processed via carding or air-laying and needle-punched, spirally wound, thermally bonded, ultrasonically bonded (all of which are known to those of ordinary skill in the art) or vertically lapped (Strudo; see, for example, U.S. Pat. No. 6,008,149, which is incorporated herein by reference). Additionally, staple fibers can be formed into a structure via chemical bonding or reinforcing of the fibers. They can also be thermally bonded in a hot-air oven or via ultrasonic techniques. The diameter of the fibers is selected largely to provide desired compression resistance. Absorbent wound packing or dressings will be finer and softer. NPWT materials will be either fine and soft or thick and much more rigid.

Another feature differentiating the present invention from the prior art is that in the present invention the method of melt-blowing the PLA fibers into continuous filaments is novel and non-obvious and imparts unique characteristics to the medical dressing of the present invention. There are many adjustment parameters in the entire melt-blowing system including temperatures, pressures, quench conditions, extrusion air velocity, suction air velocity, etc. Utilizing these process parameters, a matt can be designed to be, for example, stiff and thin or flexible and fluffy as well as producing various structures in between. Fiber diameters can range from approximately 1 to 1000 microns (μm) and it is possible to produce sub-micron fibers via increased hot air attenuation and/or low polymer throughputs. Different diameters can be achieved by adjusting the internal nozzle diameters and/or air velocity at certain nozzles or by directing external cooling air toward certain fiber streams. Finally, the incorporation of antimicrobial and other actives, polymer additives and modifiers in-situ to the meltblown process allows the “dialing in” of specific mechanical properties (moisture vapor transmission rate, tensile strength, etc.) for the PLA dressing targeted for manufacturing in this invention. The unique characteristics allow for the incorporation of multiple layers of fibers and filaments that serve specific functions including, but not limited to, three-dimensional structures or formed layers using pattern forming techniques. The multiple layering (i.e., a layered structure) is also useful to provide specific absorbency without the need to perform separate lamination operations, as is typically done in the prior art. Separate lamination operations encompasses a sequence of discrete process steps wherein sheets and webs are created on separate forming stations or machines and then utilizing a bonding system, the individuals webs are thermally or adhesively or ultrasonically fused together.

In another embodiment of the present invention, the PLA fibers of the present invention can be used in combination with other fibers such as spun-bond polypropylene or polyethylene, but the fibers used with the PLA fibers of the present invention are not limited to those two materials. Additionally, hydrophilic or hydrophobic layers in a single layer or multilayer construction are possible where either the PLA or the other polymer, or both, are treated with materials to render the nonwoven filaments hydrophilic or hydrophobic, depending on the end use and purpose (see, below, paragraph [00115). The hydrophilic and hydrophobic materials can be introduced in the fiber prior to extrusion via master-batching or via a subsequent process such as coating, spraying or dipping. The introduction of hydrophilic and hydrophobic materials to the fibers is not limited to the techniques mentioned here but can be accomplished by any technique available to those of ordinary skill in the art.

In some embodiments, fiber-reinforced layers may be prepared using composite fibers such that the fibers' core provides strength and rigidity while coatings on the fibers provide moisture holding or gelling ability. The absorbent outer structure can be applied, when the fibers are formed during a secondary process, which is generally preferred. Alternatively, it is also possible to include a thermoplastic moisture sensitive polymer into the mix such as polyoxyethylene (polyethylene glycol) while extruding the fibers.

In some embodiments, the fibers can also be core-shell type fibers, where the inner core is a polymer fiber of one type such as one that provides strength to the fiber, and the outer shell or sheath represents another polymeric material such as one that is moisture absorbent and/or has gelling properties. Core-shell types of fibers may be made in a variety of combinations of natural, naturally-derived, and synthetic polymers.

In some embodiments, the fibers can be coextruded to provide a low-melt outer surface for thermal bonding. The outer surface can also be used to deliver “actives” such as antimicrobials that elute from the fiber surface. Antimicrobials, active ingredients, or materials that assist degradation, can be “master batched” into the polymer melt and extruded with the fibers. Thus, in some embodiments, the entire fiber structure, not just the periphery of the fiber, can be used to deliver active ingredients.

In other embodiments, the fiber structure can also be hollow. The hollow structure can be modified by varying wall thickness, inside diameter of the fiber, and outside diameter of the fiber. The dimensions of the hollow fiber can be tuned, for example, to allow for increased surface area, porosity, absorbency, moisture vapor transmission rate, compression resistance, tensile strength, and active ingredient release rate.

In some embodiments, the nonwoven fibers may be further exposed to a coating process. Such processes are known in the art and include, but are not limited to, roll coating, gravure coating, gravure printing, roto press printing, slot die coating, spraying, dipping, saturating, kiss coating, partial saturation coating, Dahlgren coating, and so on. Multiple coatings can be applied in-line or in subsequent processes. The coating need not have total fiber coverage, and may be surface-oriented and/or pattern coated. In some embodiments, one side only of a nonwoven fibrous web may be treated. In some other embodiments, both sides may be treated.

Coating may be used for a variety of reasons such as a) to vary the hydrophilic/hydrophobic nature of the structure, b) to provide fluid holding capacity if desired, c) to contain and deliver a fragrance, “active” drug or antimicrobial, or d) to contain some material that will assist the degradation or biodegradation of the fibers. The hydrophilic and hydrophobic coating(s) could also be biocompatible and bio-resorbable. These coatings can be selected from, but not limited to: cellulose (hydrophobic), collagen (hydrophilic), alginate (hydrophilic), chitosan (hydrophilic), gums (hydrophobic), starch (hydrophilic), ethylene glycol species (hydrophilic), propylene glycol species (hydrophilic), polyoxyethylene (hydrophilic), polylactic acid (hydrophobic), polyhydroxyalkaonates (PHA's) (hydrophobic), polyglycolic acid their co-polymers (hydrophilic), and blends thereof. The hydrophobicity/hydrophilicity of these coating materials can be adjusted by utilizing blends. Further, some can be chemically modified to adjust and/or change the hydrophobicity/hydrophilicity, as is known to one of ordinary skill in the art. The coatings can include antimicrobial active ingredients such as, but not limited to, silver or silver-species and iodine and iodine-species. The coatings can also include chemical systems necessary for the delivery of antimicrobial species.

In some embodiments, the fibrous scaffold or backing may be coated with a full surface coating. Certain embodiments of this coating can also be mixed or injected with air or a gas, including water or steam, to reduce density and provide mechanical pores and wicking channels. The gas can be generated in-situ chemically or generated and frothed immediately prior to application. Effervescent gas-generating chemistry that reacts in the drying and/or curing phase may be advantageously used in the manufacturing process. The coating is dried, cured and generally solidified before use. In some embodiments, the structure may be cross-linked for greater integrity and strength, especially if the coating has the ability to swell and form a gel.

The extruded fibers can be any denier or Tex, both terms defined as the mass of the filament or fiber in grams of 9,000 meters or 1,000 meters respectively, and are known to those of ordinary skill in the art. The extruded fibers can also range from a minimum diameter of 1 micron to a maximum diameter of 100 microns. The fibers can be additionally processed to create more porosity, structure, and fluid-holding capability.

In our invention for the non-woven material layer, PLA fibers may be thermally glazed (calendered). Heat applied with calender rollers and even exposure to blasts of hot air, can provide the nonwoven filaments, which may comprise the entire non-woven web material with a smooth film-like surface. Still the non-woven layer may still have porosity to fluids and moisture and the porosity can also be controlled by, for example, the speed and temperature of the process. Fiber glazing process may be used instead of application of film, and provides a unique and advantageous method to control fluid flow in the nonwoven fibers, with a minimum of lamination and processing effort. Glazing can be applied as a treatment on an overall surface of fibers or various areas of the non-woven layer. This glazing or calendering process creates, in one embodiment, a semi-permeable layer that over-lays the non-woven material.

Porosity and mechanical tensile strength can be controlled by controlling the heat used to calender the material and by the usage of an engraving roll that can place apertures on the film. Glazing can be an overall surface treatment or a variable/zone application. For purposes of visual comparison only, and not for comparison to mechanical or end-use properties, the smooth glazed PLA fibrous surface resembles in looks only the commercial product Tyvek®. The purpose of the fiber glazing (calendering) process is to eliminate the need for a separate film, and it provides a unique and advantageous method to control fluid flow in the non-woven layer with a minimum of lamination and processing effort while increasing the utility of the non-woven layer. Non-limiting examples of the range of porosity and mechanical tensile strength that can be achieved by the calendering process of the present invention are shown in exemplifications below. One of ordinary skill in the art would be able, with guidance from the teachings of the present invention, to extrapolate times and temperatures necessary for a desired porosity. In one embodiment, the moisture-vapor transmission ratio of the semi-permeable layer is from about 1 to about 1000 g/hr-m² (grams per hour meter squared).

In another embodiment, nonwoven layer can be made eliminating the need for glues and adhesive bonding and, at the same time provide, if needed, perforations that allow the biological fluids to flow into an absorbent layer. The PLA glazed surface can be treated with hydrophilic and/or hydrophobic materials (see, paragraph [00115]) to help reduce adhesion to the wound and control fluid flow. Additionally, an adhesive surface can be applied including a gentle release gel adhesive that may include silicone gel or oil.

In some embodiments, the glazing provides a film-like outer surface with a fibrous inner structure. The film-like outer surface can be perforated, preferably via ultrasonic perforation, to provide various size channels and orifices for controlling fluid flow and adsorption. An engraved roller may also be used in the calendering process. Perforation may also be used as a means of bonding the PLA nonwoven structures to other structures. These other structures can be, but are not limited to, synthetic films, fibers, composites or foams, natural films, fibers, composites or foams, or naturally-derived films, fibers, composites or foams. Ultrasonic bonding and ultrasonic perforating, or roller bonding and roller perforation, both may be used to provide a bond between similar and dissimilar structures including but not limited to film to film, film to fiber, and fiber to fiber, generally employing thermoplastic materials, or materials of natural, naturally-derived, or synthetic origin, both organic and inorganic in nature.

Needle-punching can also be used advantageously to bond similar and dissimilar structures including but not limited to film to film, film to fiber, and fiber to fiber, generally employing thermoplastic materials, or materials of natural, naturally-derived, or synthetic origin, both organic and inorganic in nature. Needle-punched nonwoven structures are created by mechanically orienting and interlocking the fibers of a meltblown, spunbonded or carded web. This mechanical interlocking of the fibers is achieved with thousands of barbed felting needles repeatedly passing into and out of the web. As the needle loom beam moves up and down, the blades of the needles penetrate the fiber batting. Barbs on the blade of the needles pick up fibers on the downward movement and carry these fibers the depth of the penetration. The draw roll pulls the batt (batting) through the needle loom as the needles reorient the fibers from a predominately horizontal to almost a vertical position. Increasing the number of needles penetrating the web, results in increased density and increases web strength.

In some embodiments, perforations in the PLA glazed non-woven material can be covered by a mesh. Such a mesh can be an integral part of the nonwoven structure, or can be used as a separate structure for use in the wound or as part of the NPWT assembly.

In some embodiments, the nonwoven fibers can be treated with plasticizers to soften the fibers and render them less brittle. Such plasticizers can be, but are not limited to, other flexible synthetic, natural, and naturally-derived polymers co-polymerized with the PLA, amorphous forms of PLA, silicone oils, surfactants, polyethylene glycols such as PEG-400 as well as other molecular weight ranges of PEG, glycol ethers, such as known in the trade as Dowanol™ (glycol ethers from Dow Chemical, Midland, Mich.), polyethylene oxide polymers and oligomers such as known in the trade as “Polyox®,” octylphenoxy polyethoxy ethanol (from Dow Chemical, Midland, Mich.), tridecyl alcohol ethoxylates of various molecular weights and ethylene oxide content, surfactants, especially long-chain surfactants, plasticizers are used to provide compatibility to the fibers and soften them. Many conventional plasticizers are known in the art that soften polymers and lower the Tg, (glass transition temperature).

Plasticization can also be nonconventional. For example, temperature stable antimicrobial or biocidal agents can be employed to soften the fibers. Such a material can be master batched in the polymer melt, or applied on post-extrusion. Also, such antimicrobials and biocidal agents can be delivered using plasticizers. Using a plasticization process, the hardness characteristics of the fibers can be controlled by, but not limited to, polymer selection, purposeful selection of plasticizer, or selection of additives, such as antimicrobial additives, which have an adjuvant plasticizer effect. The plasticizers can be hydrophilic or hydrophobic.

Suitable examples of plasticizers, lubricants and processing aids are CP-L01 from Polyvel (Hammonton, N.J.) which is a PLA plasticizer specifically targeted to improving the toughness, impact and processing capabilities of PLA. Another product by Polyvel is CT-L01, a lubricant, which improves slip characteristics while retaining other properties; it decreases PLA's high coefficient of friction and therefore reduces or eliminates adhesion between other film or metal surfaces during production. Additionally, Polyvel CT-L03 is a processing aid which raises intrinsic viscosity of PLA providing increased molecular weight and improved melt strength. Finally, Polyvel HD-L02 is a rubberizer which allows for the increase in the expansion capabilities of PLA. Many other similar products are present in the commercial polymer additive and modifier marketplace.

In some other embodiments, antimicrobial agents may be delivered to the wound. The definition of an antimicrobial according to Stedman's Medical Dictionary, 26^{th} edition, 1995 is “Tending to destroy microbes, to prevent (or inhibit) their multiplication or growth, or to prevent (or inhibit) their pathogenic action.” In preferred embodiments, silver or silver-species, iodine or iodine-species may be used.

It is preferred to place “actives” within the polymer by melt blending (as described and exemplified throughout the present specification) thus, impregnating each fiber fully and/or partially. Traditionally, actives have been defined as chemical or physical agents that impart specific performance characteristics (as opposed to merely physical characteristics) to polymers. For example, it is current state of the art to incorporate into textile products actives using specialized pharmaceuticals and natural and botanical ingredients to provide odor control. In our invention, actives such as antimicrobial ingredients which mitigate and control the propagation of pathogens (and in doing so, control odor) in and on the polymer fibers and in the wound environment. A good overview of antimicrobial actives for textile application can be seen in “Recent Advances in Antimicrobial Treatments of Textiles, Yuan Gao and Robin Cranston, Textile Research Journal 2008; 78; 60” or the use of antimicrobial actives as agents in polymers in “U.S. Pat. No. 5,906,825, Polymers containing antimicrobial agents and methods for making and using same,” both of which are indicative of what is known by one of ordinary skill in the art are incorporated herein by reference.

However, many materials will not tolerate the heat and pressure of extrusion. For example, halogens (iodine, chlorine, bromine) and their salts or byproducts such as chlorides from PVC can release corrosive gas that can rapidly attack the machinery and require expensive alloys for protection; however, silver does not present these problems. As an alternative to a polymer-additive, after the polymer fibers are formed, the PLA fibers can be treated by coating, immersion, spraying, printing or any other technique capable of transferring an ingredient or ingredients onto the fibers. The purpose of such treatment could be to impart enhanced availability, and may include, but is not limited to, water, lactic acid, lactide, organic and inorganic acids and bases, and catalysts.

This invention utilizes, but is not limited to, mechanisms of action generated in situ upon contact of the pathogen with the antimicrobial agent. The in situ, contact-based action of the present invention can be controlled via reaction chemistry or a triggering event, such as contact with moisture or wound exudate, or it can be sustained released thereby providing antimicrobial and/or antifungal protection.

The antimicrobial agents of the present invention can function in the condensed phase, where condensed phase means a liquid or solid, or in a gaseous phase and said antimicrobial agents can be generated in situ via a chemical reaction, or used as-is, or released in a controlled fashion.

One novel and unique improvement of the present invention over the related prior art is the simplicity of the present invention which integrates the antimicrobial compound as a masterbatch directly into the thermoplastic (e.g., polylactic acid) fibers as part of the meltblown fiber manufacturing process with specifically tuned process variables (as exemplified below) resulting in the non-woven material used in the medical dressing product. An additional improvement of this invention is the ability to modify the calendering process (as a function of speed, pressure and temperature) of the polylactic acid polymer non-woven material with the antimicrobial formulation affording a unique platform as a medical dressing.

In some embodiments, silver species that are active against antibiotic-resistant bacteria, such as Methicillin-Resistant Staphylococcus aureus (MRSA) and Vancomycin-Resistant Enterococci (VRE) species. Silver agents are particularly attractive to providing a broad spectrum of antimicrobial activity at low concentrations with minimal toxicity toward mammalian cells. Also, silver species have a lower tendency than antibiotics to induce resistance by targeting simultaneously multiple bacterial sites.

An antimicrobial agent refers to a chemical substance that kills or inhibits the growth of bacteria, fungi, and yeasts or protozoans, that is all the various types of microbial flora present in a wound at any stage of wound healing or any stage of wound deterioration, including, but not limited to, normal skin flora, aerobic and anaerobic gram negative bacteria, and aerobic and anaerobic gram positive bacteria, including cells that form on surfaces, especially on objects, implants, scaffolds, and structures inside the body, generally called biofilms.

A preferred antimicrobial and antifungal agent is ionic silver, being released from a nonwoven layer material made preferably from PLA fibers.

Examples of suitable silver and silver ion-based agents include, but are not limited to, silver halides, nitrates, nitrites, selenites, selenides, sulphites, sulphates, sulphadiazine, silver polysaccharides where such polysaccharides include simple sugars to polymeric and fibrous polysaccharides, silver zirconium complexes, forms including organic-silver complexes such as silver trapped in or by synthetic, natural or naturally-derived polymers, including cyclodextrins; all compounds, inorganic or organic, that contain silver as part of the structure, where such structures can exist as a gas, solid, or liquid, as intact salts, dissolved salts, dissociated species in protic or aprotic solvents and silver species which contain the molecular morphology or macroscopic properties of materials in contact with silver whereby such materials, either organic, inorganic, and/or of biological nature, are found in various morphologies, such as crystalline or amorphous forms, or optical activities, such as d, I, or meso forms, or tacticities such as isotactic, atactic, or syndiotactic, or mixtures thereof.

The definition of silver species includes combinations of one or more of the above compositions, and includes such compositions being in a number of various physical forms or combinations of physical forms, such as, but not limited to, sheets, fibers, liquids, gases, gels, melts, beads, and the like. The definition also includes nano structures, which currently is taken to mean an entity or structure with at least one dimension between 1 and 100 nanometers in size. That is, both the silver or silver species is in nanomaterial form, or the entity the silver or silver species is interacting with, or combined with, is in nanomolecular form, or both the silver and silver species and the material it is interacting with is in nanomaterial form.

The term “silver” herein represents atomic silver, ionic silver, Ag, metallic silver, elemental and atomic number 47, in all its oxidation states, ionization states, or isotopic forms, including any radioactive isotopes, or mixtures thereof, and physical forms, including crystal structures and morphology. The term “silver species” means all compounds, inorganic or organic, that contain silver as part of the structure, where such structures can exist as a gas, solid, or liquid, as intact salts, dissolved salts, dissociated species in protic or aprotic solvents, and can be covalently bound, ionically bound, or bound by other mechanisms known as “charge-transfer” complexes. The definition also includes clathrate compounds (a chemical substance consisting of a lattice that traps or contains molecules) that involve silver or silver species as part of the structure, and also includes silver or silver containing species that exist as a result of the process of sorption, either chemical or physical sorption, meaning absorption or adsorption, where the sorptive surface can be a molecule, polymer, organic or inorganic entity such as, but not limited to, synthetic oligomers or polymers, either thermoplastic or thermoforming, natural or naturally-derived polymers, either thermoplastic or thermoforming, biodegradable and non-biodegradable polymers, either thermoplastic or thermoforming, and inorganic or organic species whose surface area provides for some sorptive effect. Examples of the latter can include, but are not limited to, charcoal, and zeolites of all chemical structures such as silica, diatoms, and other high-surface area materials. The definition also includes silver or silver species in all its known valence states, either organically or inorganically bound, and includes organic or inorganic materials, either gas, liquid, or solid, where the silver or silver species can “exchange” or transfer by mechanisms such as, but not limited to, ion-exchange, diffusion, replacement, dissolution, and the like including silver glass, silver zeolite, silver-acrylic and nano-silver structures. Zeolite carrier based (the silver ions exchange with other positive ions (often sodium) from the moisture in the environment, effecting a release of silver “on demand” from the zeolite crystals) and glass based silver chemistries (soluble glass containing antimicrobial metal ions wherein with the presence of water or moisture, the glass will release the metal ions gradually to function as antimicrobial agents), are non-limiting examples of silver-ion-based agents suitable for use in the present invention.

Common forms of silver that we employ or could employ in this invention include, but are not limited to silver glasses such as CorGlaes Ag® from Giltech Limited (Ayr, United Kingdom) or Ionpure® glass from Ishizuka Glass (Iwakura-shi, Aichi, Japan), liquid silver/acrylic Silvadur® from Dow Chemical Company (Spring House, Pa.), nano-silver SmartSilver® from NanoHorizons (Bellefonte, Pa.), silver zeolite structures such as those offered by Agion Incorporated (Cambridge, Mass.) or silver zirconium complexes such as those offered by Milliken (Spartanburg, S.C.). Other forms include organic-silver complexes such as silver trapped in or by synthetic, natural or naturally-derived polymers, including cyclodextrins. The silver can be utilized in the form of fibers, gels, including hydrogels, composites and foams, films, hydrocolloids, and superabsorbents. Silver is a useful material and can be associated, complexed, or bound to organic and inorganic materials, and such a list constitutes a partial cataloging of silver's use and utility. Silver, and in particular the ions of silver (Ag+, Ag++ and Ag+++) are used to reduce bacterial and fungal populations and prevent reproduction of the same. In certain studies, silver ions have been shown to control viral populations. Although the speed of control or kill is slow, hours and days, it is a powerful tool in the prevention of cross contamination, odor control and material protection. Protection can last for months or years depending on the formulation and concentration. In this application, silver may be formulated to deliver ions rapidly constantly over the use of the product and will impart an infection-control feature in a wound dressing where infections are rampant and exceptionally difficult to control.

Any combination of the above exemplary silver and silver ion-based agents is also contemplated for use in the PLA non-woven material.

In a preferred embodiment of the present invention for the PLA non-woven material, the antimicrobial and antifungal agents are incorporated into the actual fibers of the PLA non-woven material. In this embodiment, the agents are added to the polymer prior to the formation of the polymer into fibers. In yet another embodiment the antimicrobial and antifungal agents are both incorporated into the actual fibers and interspersed between the fibers.

In other embodiments, non-silver and non-silver ion-based antimicrobial and antifungal agents are contemplated for use in the non-woven layer of the present invention. These non-silver and non-silver ion-based agents may be used independent of or in conjunction with the silver and silver ion-based agents of the present invention. One of ordinary skill in the art, based on the teachings of this present specification, can determine suitable combinations of agents depending on the fiber composition of the non-woven material. Suitable non-silver and non-silver ion-based agents include, but are not limited to, compounds containing zinc, copper, titanium, magnesium, quaternary ammonium, silane (alkyltrialkoxysilanes) quaternary ammonium cadmium, mercury, biguanides, amines, glucoprotamine, chitosan, trichlocarban, triclosan (diphenyl ether (bis-phenyl) derivative known as either 2,4,4′-trichloro-2′ hydroxy dipenyl ether or 5-chloro-2-(2,4-dichloro phenoxyl)phenol), aldehydes, halogens, isothiazones, peroxo compounds, n-halamines, cyclodextrins, nanoparticles of noble metals and metal oxides, chloroxynol, tributyltins, triphenyltins, fluconazole, nystatin, amphotericin B, chlorhexidine, alkylated polethylenimine, lactoferrin, tetracycline, gatifloxacin, sodium hypophosphite monohydrate, sodium hypochlorite, phenolic, glutaraldehyde, hypochlorite, ortho-phthalaldehyde, peracetic acid, chlorhexidine gluconate, hexachlorophene, alcohols, iodophores, acetic acid, citric acid, lactic acid, allyl isothiocyanate, alkylresorcinols, pyrimethanil, potassium sorbate, pectin, nisin, lauric arginate, cumin oil, oregano oil, pimento oil, tartaric acid, thyme oil, garlic oil (composed of sulfur compounds such as allicin, diallyl disulfide and diallyl trisulfide), grapefruit seed extract, ascorbic acid, sorbic acid, calcium compounds, phytoalexins, methyl paraben, sodium benzoate, linalool, methyl chavicol, lysozyme, ethylenediamine tetracetic acid, pediocin, sodium lactate, phytic acid, benzoic anhydride, carvacrol, eugenol, geraniol, terpineol, thymol, imazalil, lauric acid, palmitoleic acid, phenolic compounds, propionic acid, sorbic acid anhydride, propyl paraben, sorbic acid harpin-protein, ipradion, 1-methylcyclopropene, polygalacturonase, benzoic acid, hexanal, 1-hexanol, 2-hexen-1-ol, 6-nonenal, 3-nonen-2-one,methyl salicylate, sodium bicarbonate and potassium dioxide.

Thus, in an embodiment of the present invention, the invention comprises a medical dressing, comprising: at least one layer (i.e., backbone layer or core) of non-woven fibers comprising one or more biodegradable thermoplastic polymers incorporating a superabsorbent agent or layer and one or more silver-based or silver ion-based antimicrobial agents incorporated into the one or more biodegradable thermoplastic polymers. The silver-based or silver ion-based antimicrobial agents are incorporated into the non-woven fibers or interspersed between the non-woven fibers. The fibers of the non-woven layer, in an embodiment, are oriented to provide expansion due to the absorption of moisture and fluids and maintain paths for liquid-flow and air-flow, preferentially in a direction transverse or essentially traverse to an exterior surface. Further, the fibers of the present invention may be vertically lapped or spirally wound. “Vertically lapped” is defined herein as meaning that the ends of one set of fibers overlap vertically with the ends of another set of fibers, i.e., the fibers of the first set of fibers and the fibers of the second set of fibers are oriented substantially in the same direction and are overlapping to some degree. “Spirally wound” is defined herein as meaning that the fibers form substantially a helix.

Polymer means natural, naturally-derived, synthetic, biopolymers, and oligomeric species thereof, with an oligomer defined as a low molecular weight polymer, which is therefore defined as a molecule having two of more repeating monomeric repeating units.

In certain preferred embodiments, the addition of protease-type de-polymerases and lipase-type de-polymerases into the polymer or fiber, to constitute a system, can also degrade the polymer.

### EXAMPLES

The following is a partial glossary and provenance of the terminology and materials used in the examples below; see Table 1. Table 1 also lists commercial suppliers of most of the recited materials.

TABLE 1

| | EXAMPLES |
|---|---|
| | Glossary |
| GDL | D-Gluconic acid δ-lactone; glucono delta |
| | lactone; gluconic acid (Sigma Aldrich, |
| | Milwaukee, WI, USA) |
| Glycerin | USP vegetable glycerin (Bulk Apothecary, |
| | Streetsboro, OH, USA) |
| HPMC | AnyCoat ® AN15; hydroxyl propyl methyl |
| | cellulose, viscosity (2 wt % aqueous solution at |
| | 20° C.) = 15.0 cps (Samsung Fine Chemicals, |
| | Korea) |
| HPMC | BENECEL ® E15; hydroxyl propyl methyl |
| | cellulose non-ionic ether, viscosity (2 wt % |
| | aqueous solution at 20° C.) = 12.0-18.0 cps |
| | (Ashland Chemical Co. Covington, KT, USA) |
| HPC | Klucel JF Pharm; hydroxyl propyl cellulose |
| | (Ashland Chemical Co. Covington, KT, USA) |
| Sodium Alginate | PROTANAL ® LF 200 FTS Sodium alginate, |
| | viscosity (1 wt % aqueous solution at 20° C.) = |
| | 200-400 cps, pH = 6.0-8.0 (FMC, Philadelphia, |
| | PA, USA) |
| 70 % Sorbitol | 70% Solution (Fisher Scientific, Waltham, MA, |
| | USA) |
| TWEEN 20 ® | Polysorbate 20; polyoxyethylene sorbitan |
| | monolaurate (J. T. Baker, Phillipsburg, NJ, USA) |
| Calcium Carbonate | ViCALity ® Precipitated calcium carbonate; |
| | ViCALity Extra Heavy (particle size, 4.5 μm), |
| | ViCality Heavy (particle size, 3.0 μm), ViCality |
| | Medium (particle size, 2.6 μm), and ViCality |
| | Light (particle size, 1.9 μm) (Specialty Minerals, |
| | Adams, MA, USA) |
| Collagen | Collagen Type I & III (NeoCell, Irvine, CA, USA) |
| HA | Proturon Std C; Hyaluronic Acid; molecular size |
| | 1.8-2.2 mill (FMC, Philadelphia, PA) |
| X-static | Silver metal coated nylon fibers (Noble |
| | Biomaterials, Scranton, PA, USA) |
| Silver Zeolite | 22% Silver Zeolite (AgIon, Wakefield, MA, USA) |
| Silver Zeolite | Ionpure WPA <5 Silver Zeolite (Ishizuka Glass |
| | Co., Japan) |
| Silver Zeolite | AC10D Silver and Copper Zeolite (AgIon, |
| | Wakefield, MA, USA) |

It is to be understood that the above detailed description of the preferred embodiments of the invention and the following exemplification is provided by way of example only. Various details of the design, construction and composition may be modified without departing from the scope of the invention as set forth in the claims. In addition, the invention will be further described by reference to the following detailed examples. These examples are merely illustrative and not limitative of Applicant's invention in any way.

### Example 1

### Method for Making the PLA Substrate Layer

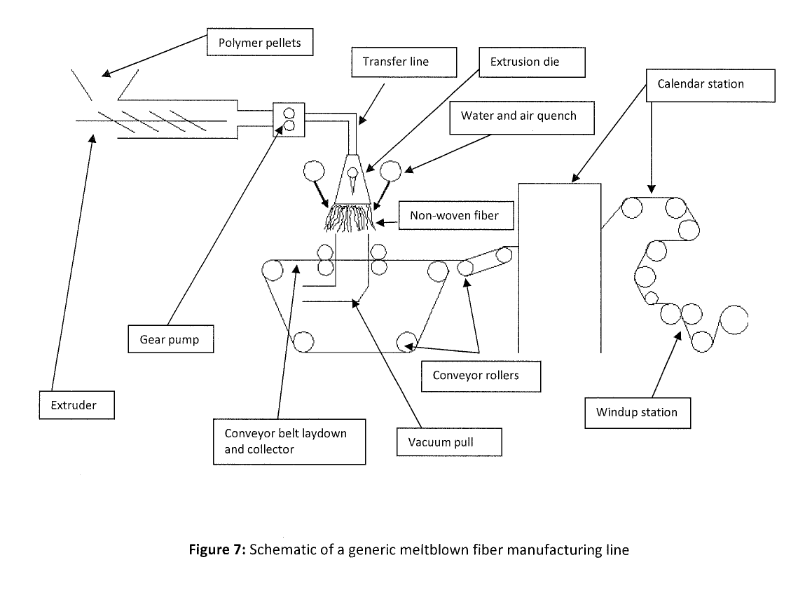

Referring to FIG. 7, Grade 6252D PLA polymer pellets from NatureWorks is utilized from a fresh unopened bag and introduced into the mouth of a 2.5″ 30:1 40-hp extruder and exposed to mechanical shear and heat ranging from 325 to 425° F. as it travels through the system. Filtration followed by a gear pump push the molten polymer thru a heated transfer line into a BIAX meltblown system at 800 to 2000 pounds per square inch (psi). Compressed air is heated to 475-525° F. and introduced into the die at 10-18 psi and used to attenuate the PLA fibers thru nozzles with an internal diameter of 0.012″. A filtered water mist quench is produced using a high-pressure piston pump and a fluid-misting system. This quench is operated at 500-1800 psi and the mist impinges the fibers as they exit the die zone and serves to cool them. An air quench system introduces cool outside air to the fibers before they are deposited on a flat belt with a vacuum source below. The speed of this belt determines the weight of the web. For most advanced wound care applications a wound absorbent non-woven layer between 10 and 1000 grams per square meter (gsm) is required. The vacuum level additionally serves to compress the web, or allow it to remain fluffy and at a low density. Calendering or thermal point bonding can serve to strengthen the wound absorbent non-woven layer and impart strength. An alternative is to place a lightweight (14-20 gsm) spunbond nonwoven fabric under the web of fibers to impart strength. Once the non-woven layer is calendered, it is directed to a windup station for final packaging and assembly.

Following collection on the belt, the web is wound into a roll and delivered to a roll wind up station. Depending on the requirements of the application, this web can be unwound from the station, and passed through a series of rollers and lamination stations, to get conjoined with an equivalent web, to yield a non-woven layer with increased compressibility and mechanical characteristics. Such a web, either one layer, or two layers or multiple layers can be conveniently cut to get converted at a later stage into finished advanced wound care products.

As a reference for mechanical properties, the tensile strength of one 33 gsm PLA layer was measured to be 0.765 in/lbs using a Thwing-Albert Tensile Tester using ASTM D5035 protocols. A 66 gsm PLA layer was measured to be 3.884 in/lbs using a Thwing-Albert Tensile Tester using ASTM D5035 protocols.

### Example 2

### Calendering Outer PLA Non-Woven Fiber Layer

In order to impart different properties to the outer non-woven PLA layer of the wound dressing, calendering can be utilized. We used a BF Perkins (division of Standex Engraving, LLC, Sandston, Va.) Calender Station which contained two heated rolls and two hydraulic rams. Each heated roll was filled with high temperature oil, which was heated by a separate machine. A hot oil machine controlled the temperature and the flow of oil through each zone of the Calender Station. The temperature can range from 110° F. to 550° F. The hot oil was circulated at 30 psi through 2 inch iron pipes into a rotary valve for each zone.

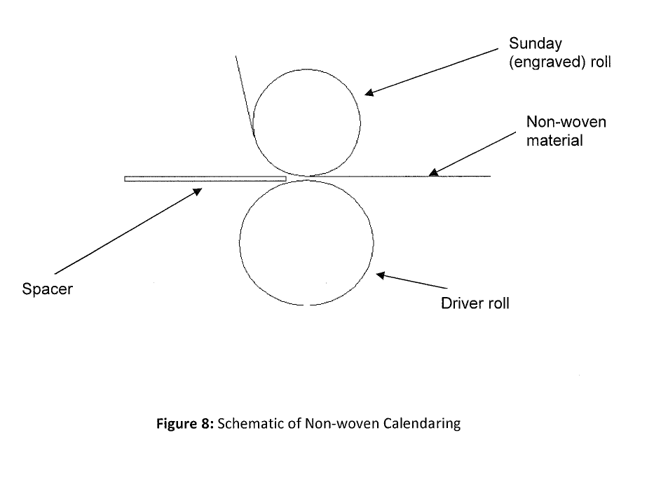

The Calender Station was opened and closed by a control station which also regulated the amount of pressure used to move the hydraulic rams. This pressure can range from 1 psi to 3,000 psi and maintained the amount of force with which the Drive Roll was supported. A variable spacer between the Sunday Roll (also called an Engraved Roll) and the Drive Roll maintained the distance of one roll to the other. The spacer allowed for the thickness of the PLA and the hydraulic rams maintain that distance. See, FIG. 8 for a schematic representation of the process. Non-limiting specifications are given below. One of ordinary skill in the art will be able to modify these specifications based on the guidance provided by this specification.
i. Top roll, labeled Sunday Roll, was an engraved roll; 7⅜″ diameter by 20″ length.
ii. Bottom Roll, labeled Drive Roll, was a smooth roll; 10″ diameter by 19½″ length.
iii. The temperature was variable on product density and speed of the process line. The speed can range, for example, from 1 to 200 FPM (feet per minute) with a temperature of 175° F. to 350° F.
iv. The distance between the rolls was a variable controlling product thickness which can range from 0.5 to 0.001 inch.

### Example 3

### Creation of Multiple PLA Medical Dressing Layers with Silver Antimicrobial

One PLA layer was laminated to another PLA perforated or apertured film created by uniquely calendering the PLA fibers to provide mechanical cushioning and antimicrobial action. The silver impregnated within the PLA film fibers is the source of antimicrobial efficacy protecting the non-woven against the propagation of bacteria, yeasts, and fungi.

1AWC-1 and 2AWC-1 are sample identifiers for manufactured PLA non-woven layer with PLA film prepared according to process specifications and properties shown in Table 2. 1AWC-1 is two layers of 50 gsm melt spun PLA integrated with a formulation of silver zeolite grade AC-10D from AgION (Wakefield, Mass.) coupled with silver glass grade WPA Ionpure® from Marubeni/Ishizuka (Santa Clara, Calif.). 2AWC-1 is two layers of 33 gsm melt spun PLA integrated with a formulation of silver zeolite grade AC-10D from AgION coupled with silver glass grade WPA Ionpure® from Marubeni/Ishizuka, each is calendered to bond the two layers of PLA melt spun. Edge sealing refers to the samples having been heat sealed on all four edges of the film structure using a standard heat sealing bar, such as ¼″ band, impulse foot sealer (American International Electric, Whittier, Calif.) at the “4” dial setting.

Table 2 is shown below:

TABLE 2

| | Line | | | | Tensile |
|---|---|---|---|---|---|
| | Speed | Temper- | Calender | | Strength |
| | (feet per | ature | Gap | Thickness | ASTM |
| Samples | minute) | (° F.) | (inches) | (inches) | D5035 |
| 1AWC-1 W/O | 20 | 240 | 0.015 | 0.019 | 10.724 in/lbs |
| Edge Sealing | | | | | |
| 1AWC-1 W/ | 20 | 240 | 0.015 | 0.019 | 10.470 in/lbs |
| Edge Sealing | | | | | |
| 2AWC-1 W/O | 120 | 280 | 0.009 | 0.016 | 3.684 in/lbs |
| Edge Sealing | | | | | |
| 2AWC-1 W/ | 120 | 280 | 0.009 | 0.016 | 3.808 in/lbs |
| Edge Sealing | | | | | |

Different variations of PLA calendered film can be manufactured with different mechanical properties. For example, PLA Film 1 is calendered 33 gsm PLA integrated with a formulation of silver zeolite grade AC-10D from AgION coupled with silver glass grade WPA Ionpure® from Marubeni/Ishizuka at 240° F., 40 feet per minute (fpm), at 0.001″ gap at about 900 psi. PLA Film 2 is calendered 66 gsm melt spun PLA integrated with a formulation of silver Zeolite grade AC-10D from AgION coupled with silver glass grade WPA Ionpure® from Marubeni/Ishizuka at 280° F., at 10 fpm, at 0.005″ gap, under 1,000 psi. The corresponding test data is shown below in Table 3.

Table 3, as shown below, reflects the significant difference in the properties for the calendered and uncalendered versions of PLA Film 1 and PLA Film 2:

TABLE 3

| | Tensile Strength | Apparent elongation |
|---|---|---|
| Samples | (ASTM D5030) | (%) |
| PLA Film 1 | 2.999 in/lbs | 6.884 |
| PLA Film 2 | 5.579 in/lbs | 5.064 |
| PLA Film 1- | 0.765 in/lbs | 5.886 |
| uncalendered | | |
| PLA Film 2- | 3.784 in/lbs | 3.814 |
| uncalendered | | |

As a reference for mechanical properties, the determination of permeation is conducted according to ASTM E96/E96M-10, Water Vapor (moisture vapor) Transmission of Materials Test methodology using permeation cups by BYK-Gardner (Columbia, Md.) and weigh scale by Mettler Toledo (Columbus, Ohio).

The size of the apertures for PLA Film 1 and PLA Film 2 were measured to be 0.022 inches in diameter. The apertures can be of a given shape (circular, diamond, etc.) as determined by the design of the engraved roll (Sunday roll).

Additional permeation characteristics can be designed with various constructions as exemplified in the Table 4 below.

Table 4 is shown below:

TABLE 4

| | Permeation |
|---|---|
| | (ASTM E96) |
| Sample Construction | (g/24 hr-m²) |
| Two layers of 50 gsm uncalendered PLA integrated with a | 156.7750 |
| formulation of silver zeolite grade AC-10D from AgION | |
| coupled with silver glass grade WPA Ionpure ® from | |
| Marubeni/Ishizuka | |
| Two layers of 66 gsm calendered PLA integrated with a | 148.0729 |
| formulation of silver zeolite grade AC-10D from AgION | |
| coupled with silver glass grade WPA Ionpure ® from | |
| Marubeni/Ishizuka with two layers of 50 gsm calendered | |
| PLA layer between the 66 gsm PLA layers | |
| Two layers of 66 gsm calendered PLA integrated with a | 157.4042 |
| formulation of silver zeolite grade AC-10D from AgION | |
| coupled with silver glass grade WPA Ionpure ® from | |
| Marubeni/Ishizuka with two layers of 33 gsm calendered | |
| between the 66 gsm PLA layers. | |

PLA calendered film can also be laminated to itself with or without heat sealing by means of a secondary a second calendering step to create a stronger or differently functional structure. When desired, heat sealing can be conducted on two edges (machine web direction or machine cross direction). Additionally, the PLA calendered films can be laminated to other PLA films and heat sealed. In Table 5 below, some of the combinations of structures and the corresponding mechanical properties are shown. The heat sealing for Table 5 was conducted in the machine web direction using a standard heat sealing bar, such as a ¼″ band, impulse foot sealer (American International Electric, Whittier, Calif.) at the “4” dial setting was used to seal the edges.

Table 5 is shown below:

TABLE 5

| | | Tensile |
|---|---|---|
| | Thickness | Strength |
| Samples | (in) | (in/lbs) |
| Two layers of Film1 sealed together. | 0.006 | 6.379 |
| Two layers of Film1 calendered together. | 0.006 | 7.652 |
| Two layers of Film2 sealed together. | 0.018 | 8.276 |
| Two layers of Film2 calendered together. | 0.019 | 10.631 |
| Two layers of Film1 and one layer of 1AWC-1 | 0.018 | 10.092 |
| sealed together. | | |
| Two layers of Film1 and one layer of 1AWC | 0.028 | >11 |
| calendered together. | | |
| Two layers of Film2 and one layer of 1AWC-1 | 0.034 | 10.664 |
| sealed together. | | |
| Two layers of Film2 and one layer of 1AWC-1 | 0.019 | >11 |
| calendered together. | | |
| Two layers of Film1 and one layer of 2AWC-1 | 0.026 | >11 |
| sealed together. | | |
| Two layers of Film1 and one layer of 2AWC-1 | 0.019 | >11 |
| calendered together. | | |
| Two layers of Film2 and one layer of 2AWC-1 | 0.042 | >11 |
| sealed together. | | |
| Two layers of Film2 and one layer of 2AWC-1 | 0.028 | >11 |
| calendered together. | | |

A variety of layers with different densities, each providing a specific performance characteristic can be stacked, calendered and constructed to provide multiple or single PLA layer(s) having differing thicknesses and size.

### Example 4

### PLA Substrate with Polymer Additives for Lubrication

In a manner similar to Example 1 and utilizing a process that Biovation has developed to reformulate or modify end use properties, a polymer additive or masterbatch in dry form is added in with the PLA directly to impart lubricity. When added to the PLA at a level of 0.5%-10%; more commonly 1%-8% and more usually 1.5-5.0%, a higher volumetric throughput rate (higher density) was observed while keeping the operating pressures same, indicating lower resistance to pumping. The higher volumetric throughput rate was observed by the increased rpm on the melt-pump and extruder motor. The melt additive used was one or more selected from the group of multipurpose plasticizer additives including but not limited to CP-L01 from Polyvel Inc., BioStrength 700 (Arkema), Paraloid BPMS-250 (Dow), and Paraloid BPMS-260 (Dow). When CT-L03 (also from Polyvel) was substituted, at the same level as recommended for a lubricant or processing aide for “slip” the same throughput rate at lower extruder and melt pump speeds was achieved. Various plasticizers may be used in place of CT-L03 including: Proviplast C-series (Proviron), Proviplast 01422, Proviplast 2624, Hallgreen R-8010 (HallStar), and Hallgreen R-9010.

The data set forth in Table 6 below, show the change in density (gsm) for different runs of PLA integrated with a formulation of silver zeolite grade AC-10D from AgION coupled with silver glass grade WPA Ionpure® from Marubeni/Ishizuka with different process settings and with different levels of additives.

Table 6 is shown below:

TABLE 6

| | Density, extruder speed (rpm) |
|---|---|
| Samples | and melt-pump speed (rpm) |
| 100% PLA non-woven | 63 gsm, Extruder RPM 12%, |
| | Melt Pump RPM 19% |
| 97% PLA non-woven with | 65 gsm, Extruder RPM 13.5%, |
| 3% CP-L01 | Melt Pump RPM 21% |
| 97% PLA non-oven with | 55 gsm, Extruder RPM 11%, |
| 3% CT-L01 | Melt Pump RPM 18% |
| 94% PLA non-woven with | 63 gsm, Extruder RPM 11%, |
| 3% CP-L01 and 3% CT- L01 | Melt Pump RPM 18% |

### Example 5

### PLA Topical Hydrophilic Treatment

This proprietary Biovation process is somewhat similar to Example 1 except that the hydrophilic additive was in liquid form mixed into the water quench system and sprayed directly onto the fibers while hot. One or more candidate surfactants were selected from the group such as Triton X-100, anionic surfactants, non-ionic surfactants, or the C₁₂ diester additives such as PEG-200 or PEG 400 are preferred with the most preferred candidate being a low molecular weight polyethylene glycol (PEG). The concentration used is based on the weight of the fibers strayed and a range of 0.05% to 2.0% has proved beneficial in promoting rapid fiber wet-out. Additionally, the resultant fibrous web demonstrated a more rapid fluid acquisition speed. This enhanced hydrophilicity is advantageous when an absorbent article with rapid fluid uptake is desired. The liquid additive used was Lurol PP-2213 from Goulston Technologies, Inc. and is marketed as a single-use surface hydrophilic agent into the hygiene and diaper industry. The results are dramatic as almost immediate wet-out occurs. Another product, Triton X-100 (Dow Chemical, Midland, Mich.) was also tried successfully. It was applied to a 3×3 inch, 33 gsm PLA non-woven layer integrated with a formulation of silver zeolite grade AC-10D from AgION coupled with silver glass grade WPA Ionpure® from Marubeni/Ishizuka, from slurry, at 1% and 0.5%. Each sample was fully submerged into a volume of water and then weighed with these results and shown in Table 7 below.

Table 7 is shown below:

TABLE 7

| | Dry Weight (g) | Wet Weight (g) |
|---|---|---|
| 0% Triton X-100 | 0.19 | 0.45 |
| 0.5% Triton X-100 | 0.19 | 1.66 |
| 1% Triton X-100 | 0.19 | 1.72 |

### Example 6

### Ionic Silver Sustained Controlled-Release

This is similar to Example 1 in all aspects except that a custom masterbatch containing a slow-release silver ion compound was incorporated to provide broad antimicrobial and antifungal performance. Several silver-releasing materials have been evaluated including, silver zeolite grade AC-10D, silver glass grade WPA, silver zirconium, AlphaSan from Milliken. In each case, a 20-30% loading in a carrier polymer was prepared and used to uniformly deliver the silver additive into the mix. One preferred silver product is the silver zeolite grade AC-10D which also contains copper elements as an anti-fungal agent. Another preferred silver zeolite is the WPA Ionpure® silver glass powder. Particle size of less-than 5 micron was specified with an average of 2-3 microns to preclude spinneret nozzle clogging. The final concentration of silver in the meltblown fibers is dependent on the quantity of masterbatch used. In trials, up to 20% zeolite masterbatch has been processed to demonstrate an extreme loading, 5% silver by weight based upon the silver contained within the zeolite. For the performance required of medical dressings, we have found 1 to 200 ppm loadings, of actual silver by weight, to be effective. In advanced wound care application, silver is highly effective as its slow release and long-term bacterial control properties match the end-use requirements. The silver can be placed in a masterbatch with PLA, or an olefin carrier. For PLA fibers, we prefer the PLA carrier simply to maintain the degradability performance. The antimicrobial action of the silver is triggered upon contact with moisture.

To determine the efficacy of antimicrobial formulation, samples of a PLA non-woven fiber layer sheet (Lot: TP05062013 with 16% of masterbatch which is 80% PLA and 20% WPA Ionpure® silver glass powder and 16% of masterbatch which is 80% PLA and 20% silver Zeolite grade AC-10D) was submitted to NAMSA (Irvine, Calif.) for testing utilizing the ASTM E2149 testing protocol with sample size of 1 g, target inoculum level of 1.5−3.0×10⁵ CFU/mL with the organisms Klebsiella pneumonia (KP) source no 4352, Staphylococcus aureus (MRSA) source no ATCC 33591, Enterococcus faecalis (VRE) source no ATCC 51575, Pseudomonas aeruginosa (PA) source no ATCC 9027, and Candida albicans (CA) source no ATCC 10231. Data acquired by NAMSA is shown below in Table 8.

Below is the test data in Table 8.

TABLE 8

| | Organism Count | Organism Count | Percent |
|---|---|---|---|
| Test Article | (CFU/mL) - | (CFU/mL) - | |
| Identification | Zero Time | 4 Hour | Reduction |
| 05062013 - MRSA | 2.30 × 10⁵ | <1.00 × 10² | >99.96 |
| Control - MRSA | 3.38 × 10⁵ | >3.00 × 10⁷ | No reduction |
| 05062013 - KP | 1.58 × 10⁵ | 4.68 × 10³ | 96.80 |
| Control - KP | 2.13 × 10⁵ | >3.00 × 10⁷ | No reduction |
| 05062013 - VRE | 3.30 × 10⁵ | <1.00 × 10² | >99.97 |
| Control - VRE | 4.30 × 10⁵ | >3.00 × 10⁷ | No reduction |
| 05062013 - PA | 2.73 × 10⁵ | <1.00 × 10² | >99.96 |
| Control - PA | 2.23 × 10⁵ | >3.00 × 10⁷ | No reduction |
| 05062013 - CA | 2.53 × 10⁵ | 1.25 × 10² | 99.95 |
| Control - CA | 3.58 × 10⁵ | >3.00 × 10⁷ | No reduction |

### Example 7

### Measuring Silver Content in PLA Non-woven Material Layer

The analysis of solid samples for elements such as silver has been much studied and each was found to have some liabilities or difficulties. Methods such as wavelength dispersive X-ray fluorescence spectroscopy (WD-XRFS), laser ablation inductively coupled plasma mass spectrometry (LA-ICPMS) as well as conventional acid digestion in a Kjeldahl flask in combination with dry ashing and microwave assisted digestion followed by atomic absorption spectrometry (AAS) are the “go to” analytical tools especially for biological and environmental samples. However, solid sample analysis affords some challenging issues for each of the aforementioned methods as described in F. Vanhaeke, et al, Spectrochimica Acta: Part B 62, (2007) pp 1185-1194. For example, this study showed LA-ICPMS has potential for the direct analysis of solid samples but for variations in ablation efficiency which affords calibration difficulties. Similar calibration issues arise with WD-XRFS, mainly due to differences in absorption efficiency of X-rays. These authors describe having obtained accurate results for Ag determination using conventional acid digestion in a Kjeldahl flask in combination with dry ashing and microwave assisted digestion followed by AAS. Occasionally however, they noted analyte losses and/or incomplete dissolution as the source(s) of discrepancy.

The reagents and materials for experimentation were as follows. As specified by good lab practice, only high purity reagents were employed in sample preparation. A Millipore (Billerica, Mass.) Milli-Q system was used to generate water of 18 MΩ purity. Concentrated nitric acid (HNO₃) and 30% hydrogen peroxide (H₂O₂) were obtained from Fisher Chemical (Houston, Tex.) and (1 mg/mL) Ag in HNO₃ was obtained from Acros Organics/Thermo Fisher Scientific (Geel, Belgium and Boston, Mass.) for sample digestion and calibration standard preparation, respectively. The non-woven material with silver antimicrobial was manufactured as exemplified in the examples above.

For the digestion of PLA non-woven samples, we used a HotBlock Pro Digestion System from Environmental Express (Charleston, S.C.). The 54-well HotBlock Pro for 50 mL samples has an external thermocouple and an external controller to monitor and record sample temperatures. The controller also allows you to program and implement the digestion method (see below). For analysis of samples by Atomic Absorption Spectrometry, an ICE 3000 Series Flame AA Spectrometer from Thermo Fisher Scientific (West Palm Beach, Fla.) was used. The silver (Ag) hollow cathode lamp was purchased separately from Thermo Fisher Scientific (West Palm Beach, Fla.)

For digestion, we employed an adaptation of EPA Method 3050B for use with the Environmental Express HotBlock Digestion System. The 0.5 g samples were each placed into a 50 mL borosilicate digestion vial to which 5 mL of a 1:1 mixture of concentrated HNO₃ and 18 MΩ water is post added. The digestion vials were placed into the HotBlock unit, affixed with reflux caps and heated at 95° C. for 15 min. Samples were allowed to cool and an additional 5 mL of concentrated HNO₃ was added and then heated @95° C. for 30 min. This step was repeated until no brown fumes were given off by the samples. The samples were then heated for an additional 1.5 hours after which they were removed from the HotBlock Pro and completely cooled. To each of these vials was added 2-5 mL of 18 MΩ water and 0.5 mL of 30% H₂O₂ slowly. An exothermic reaction was allowed to occur for approximately 5-10 minutes and the samples were placed back in the HotBlock with the ribbed watch glasses in place. Effervescence was controlled by lifting the samples out of the HotBlock while allowing the reaction to continue. Care was taken to ensure that the samples did not overflow the vials. H₂O₂ was continually added in 0.5 mL increments until the sample remained unchanged in color (no longer than 30 minutes). Then heating was continued for a total of 2 hours.

For the analysis of samples for Flame AA, 5 mL of concentrated hydrogen chloride (HCl) was added to each sample and covered with a ribbed watch glass and heated to reflux at 95° C. for 15 minutes. After cooling completely, the samples were diluted to 50 mL with 18 MΩ water. A calibration curve was constructed on the basis of absorbance obtained for aqueous standards containing 0.5 ppm, 10 ppm, and 50 ppm Ag in solution.

Two identical sets of samples were tested to account for repeatability; they are denoted as “A” and “B” in the testing protocol.

The sample weights and composition of materials is shown in Table 9 below. MB21 is a master-batch with of 20% silver zeolite grade AC-10D from AgION with 80% PLA; whereas MB23 is a masterbatch with 20% silver glass grade WPA Ionpure® from Marubeni/Ishizuka with 80% PLA.

TABLE 9

| | | Weight | Weight | Weight of |
|---|---|---|---|---|
| Sample | | of A | of B | Previous |
| # | Sample Information | (g) | (g) | (g) |
| 1 | Control PLA non-woven | 0.50 | 0.51 | 0.49 |
| 2 | 97% PLA with 3% MB21 | 0.51 | 0.50 | 0.48 |
| 3 | 92% with 8% MB21 | 0.49 | 0.51 | 0.50 |
| 4 | 98.5% PLA 1.5% MB23 | 0.50 | 0.50 | 0.48 |
| 5 | 96% PLA with 4% MB23 | 0.51 | 0.49 | 0.49 |
| 6 | 92.25% PLA with 4% MB21 & | 0.50 | 0.51 | 0.50 |
| | 1.75% MB23 | | | |

The results obtained from the analysis of these samples run in triplicate are presented in Table 11. These results are expressed in ppm Ag. The expected Ag content, presented in Table 10, has been calculated based upon the type of silver (WPA Ionpure® or AgION) and the amount added during processing. We observed good agreement between the theoretical values and the analytical results with the exception of samples 4 & 6. Sample 4 is lower than the lower end of the theoretical range in 2 of the 3 repeat samplings, while sample 6 is a bit higher than the high end of the range for all three repeat samplings.

Table 10 is shown below for theoretical Ag calculations. Because the silver zeolite (AgION) has a range of 2%-5% pure silver content, the theoretical calculations for Samples 4-6 are denoted for 2% and 5% levels individually.

TABLE 10

| Sample # | Concentration A (ppm) | | Concentration B (ppm) | | Prev. Concentration (ppm) | |
|---|---|---|---|---|---|---|
| 1 | 0 | | 0 | | 0 | |
| 2 | 1.0 | | 0.96 | | 0.92 | |
| 3 | 2.51 | | 2.61 | | 2.5 | |
| 4 | (2%) 0.50 | (5%) 1.5 | (2%) 0.50 | (5%) 1.5 | (2%) 0.5 | (5%) 1.4 |
| 5 | (2%) 1.63 | (5%) 4.08 | (2%) 1.57 | (5%) 3.92 | (2%) 1.5 | (5%) 3.9 |
| 6 | (2%) 1.9 | (5%) 2.9 | (2%) 2.02 | (5%) 3.1 | (2%) 1.9 | (5%) 2.9 |

Table 11 is shown below for Ag determination by Flame AA.

TABLE 11

| Sample | Conc. A (ppm) | Conc. B (ppm) | Prev. Conc. (ppm) |
|---|---|---|---|
| 1 | 0.0117 | 0.001 | 0.009 |
| 2 | 1.0938 | 1.0111 | 0.987 |
| 3 | 3.1407 | 3.3181 | 2.763 |
| 4 | 0.3606 | 0.3770 | 0.513 |
| 5 | 2.9912 | 2.5286 | 3.126 |
| 6 | 5.9774 | 6.0543 | 4.906 |

The data indicates that the present invention for the non-woven material layer can have a lower percentage of silver content than what is commonly in the marketplace (80 to 400 ppm) to deliver equivalent level of antimicrobial efficacy as exemplified above resulting in a product that is more cost-efficacious.

From all the samples which we have run, we tend to think that these out of range values are likely variability due to material handling and process conditions.

### Example 8

### Substrate Layer Made from PLA with Polycaprolactone Resin

This is similar to Example 1, above, with the exception that Polycaprolactone (PCL) was added to the PLA in a blend at various levels from 5% to over 70%. PCL is a naturally derived polymer with a very low melt point. When used at low levels, generally 30% and lower, it functions as a plasticizer for the PLA, a brittle polymer, and imparts lubricity and softness to the fibers that functions to reduce breakage. This dramatic improvement is apparent even at a 2% add-on level and increases with concentration. The PLA/PCL blend can also incorporate masterbatch additives or surface finishes to control surface hydrophilicity and fluid wet-out. Silver can also be incorporated. The lower processing temperature of the PCL allows the use of low-temp additives but also limits the effective storage and use temperatures of the finished product.

Table 12, as shown below, reflects the mechanical properties of various PLA/PCL structures. For example, PLA/PCL Structure UC-1 is non-calendered 600 gsm 93% PLA with 1.5-5.0% CP-L01 and 1.5-5.0% CT-L03 and 0.1-2% PCL run at 400° F., 3 fpm and 1100 psi. Corresponding test data is shown below for various combinations wherein the speed, pressure and temperature were also changed.

Table 12 is shown below:

TABLE 12

| | Tensile | Apparent | |
|---|---|---|---|
| | Strength | elongation | Break Time |
| | (ASTM D5035) | (%) | (sec) |
| PLA/PCL Structure UC1 | 0.732 | 28.996 | 4.375 |
| PLA/PCL Structure UC2 | 0.937 | 14.131 | 2.141 |
| PLA/PCL Structure UC3 | 1.109 | 16.356 | 2.547 |
| PLA/PCL Structure UC4 | 1.837 | 12.024 | 1.843 |
| PLA/PCL Structure UC5 | 1.731 | 21.465 | 3.313 |
| PLA/PCL Structure UC6 | 1.347 | 22.304 | 3.391 |
| PLA/PCL Structure UC7 | 1.840 | 23.915 | 3.609 |
| PLA/PCL Structure UC8 | 1.360 | 10.460 | 1.594 |
| PLA/PCL Structure UC9 | 1.375 | 18.804 | 2.844 |
| PLA/PCL Structure UC10 | 1.767 | 17.139 | 2.734 |
| PLA/PCL Structure UC11 | 1.730 | 25.954 | 4.000 |
| PLA/PCL Structure UC12 | 1.316 | 21.022 | 3.250 |
| PLA/PCL Structure UC13 | 0.797 | 22.914 | 3.469 |
| PLA/PCL Structure UC14 | 1.176 | 15.248 | 2.312 |
| PLA/PCL Structure UC15 | 0.755 | 27.581 | 4.157 |
| PLA/PCL Structure UC16 | 0.851 | 19.247 | 2.906 |
| PLA/PCL Structure UC17 | 1.205 | 20.022 | 3.094 |
| PLA/PCL Structure UC18 | 1.118 | 23.247 | 3.562 |

The mean is 1.277 lbs for tensile strength, 20.046% for apparent elongation and 3.063 sec for break time.

By calendering various samples, the following data shown in Table 13 was obtained:

Table 13 is shown below:

TABLE 13

| | Tensile | Apparent | |
|---|---|---|---|
| | Strength | elongation | Break Time |
| | (ASTM D5035) | (%) | (sec) |
| PLA/PCL Structure 1 | 1.957 | 18.478 | 2.797 |
| PLA/PCL Structure 2 | 1.636 | 15.690 | 2.468 |
| PLA/PCL Structure 3 | 1.702 | 16.475 | 2.500 |
| PLA/PCL Structure 4 | 1.621 | 14.251 | 2.157 |
| PLA/PCL Structure 5 | 1.357 | 12.808 | 1.937 |
| PLA/PCL Structure 6 | 2.032 | 12.911 | 1.953 |
| PLA/PCL Structure 7 | 1.117 | 23.799 | 3.593 |
| PLA/PCL Structure 8 | 1.481 | 10.696 | 1.704 |
| PLA/PCL Structure 9 | 2.268 | 19.359 | 3.000 |
| PLA/PCL Structure 10 | 2.221 | 17.755 | 2.750 |
| PLA/PCL Structure 11 | 2.185 | 22.342 | 3.375 |

The mean is 1.780 lbs for tensile strength, 16.779% for apparent elongation and 2.567 sec for break time.

### Example 9

### Influence of Fiber Diameter on Performance

By varying the throughput rate of the molten polymer and the air used for attenuation, the fiber diameter and degree of polymer orientation within the fiber may be modified. Additionally, the internal diameter of the polymer nozzles, in the die or spinneret plate can be modified. In this example the polymer and thru put rate was held constant while spinneret plates with different diameters were utilized and the effect of fiber diameters was measured. Extruder zone temperatures, die-head temperatures and pressures, collector belt speed and quench air settings were optimized. Nozzle diameters ranging from 0.011 to 0.023 inches were evaluated and resultant changes in fluid management and physical cushioning were observed.

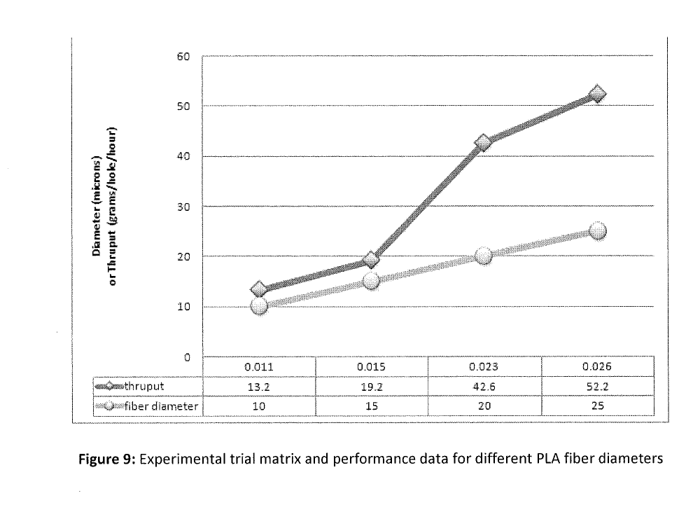

An experimental trial matrix and performance data are shown in Table 14 below and plotted as shown in FIG. 9:

Table 14 is shown below:

TABLE 14

| Throughput | g/hole/hour | 13.2 | 19.2 | 42.6 |
|---|---|---|---|---|
| Fiber Diameter | microns | 10 | 15 | 20 |
| Nozzle ID | inches | 0.011 | 0.015 | 0.023 |

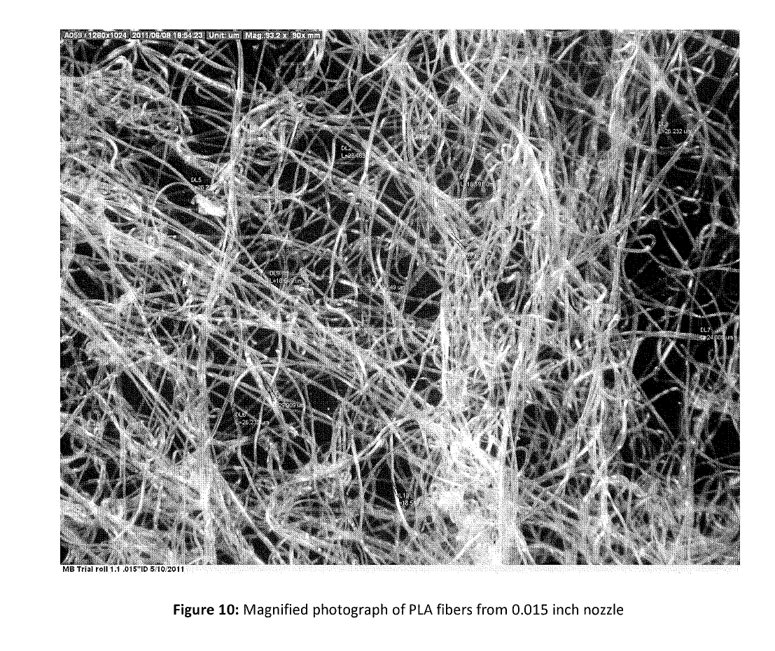

Magnified photograph of fibers from 0.015 inch nozzle, yielding a 0.015 micron diameter (average measurement of 10 fibers with a standard deviation of 4 microns) fiber is shown in FIG. 10.

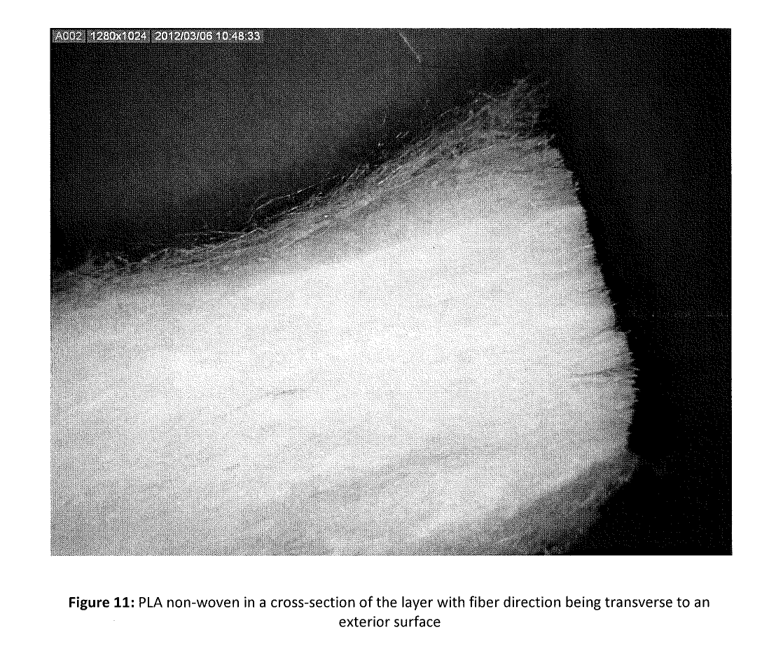

Magnified photograph of fibers from 0.015 inch nozzle showing the PLA non-woven in a cross-section of the layer with fiber direction being transverse to an exterior surface; also film orientation wherein the top surface is the horizontal surface on the photograph, and the side of the insert is the vertical surface as shown in FIG. 11.

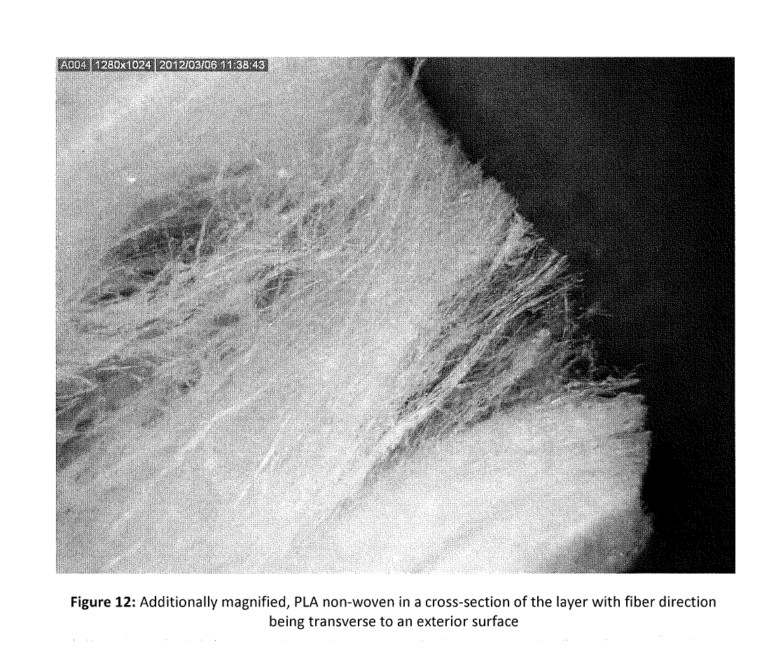

Magnified photo of fibers from 0.015 inch nozzle showing the PLA non-woven in a cross-section of the layer with fiber direction being transverse to an exterior surface; the partially vertical surface is the side of the layer, in an even more magnified photograph is shown in FIG. 12.

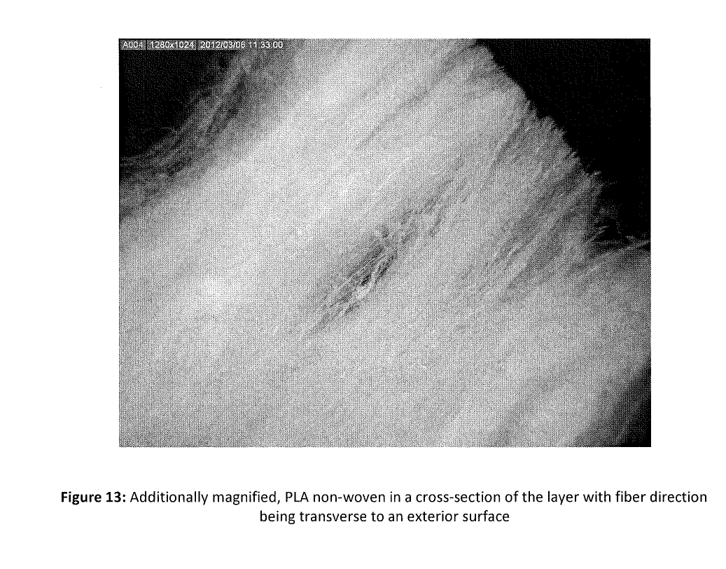

Magnified photo of fibers from 0.015 inch nozzle showing the PLA non-woven in a cross-section of the layer with fiber direction being transverse to an exterior surface; the partially vertical surface is the side of the insert, in an even more magnified photograph is shown in FIG. 13.

### Example 10

### Non-Woven Fiber Material Made with Polypropylene Resin

This is similar to all above examples with the exception of polypropylene polymer (PP) is substituted for the PLA. The advantage of PP is a higher processing and throughput speed. PP has all the required health and safety and low-bioburden properties medical dressings require. It is also receptive to hydrophilic additives in a masterbatch or surface treatment to impart rapid fluid wet-out. Additives can also be easily included in masterbatch form. A PP meltblown web can also be thermally point bonded or placed on a spunbond carrier for additional strength and can be processed in a secondary treatment step to impart a silver-containing treatment.

In this example, we used ExxonMobil (Houston, Tex.) Achieve 6936G ultra-high melt flow rate polypropylene at the 100% level and with additives. One distinct advantage was lower melt processing conditions when compared to PLA. Resultant extruder and spinning temperatures in the 275-350° F. range were sufficient to be able to utilize heat-intolerant polymer additives.

The following table (Table 15) shows the particulars of a 3BSK-1 all PP sample manufactured on the meltblown line. 3BSK-1 consists of two 50 gsm PP melt spun layers and 25 gsm of SAP, calendered to bond the SAP between the two layers of PP. Edge sealing refers to the samples heat sealed on all four edges of the film structure using a standard heat sealing bar, such as a ¼″ band, impulse foot sealer (American International Electric, Whittier, Calif.) at the “4” dial setting.

Table 15 is shown below:

TABLE 15

| | Line | Temper- | Calender | Thick- | Tensile Strength |
|---|---|---|---|---|---|
| | Speed | ature | Gap | ness | (ASTM D5035) |
| | (ft/min) | (F.) | (in) | (in) | in/lbs |
| BSK-1 W/O | 10 | 250 | 0.005 | 0.019 | 5.65 |
| Edge Sealing | | | | | |
| BSK-1 W/ | 10 | 250 | 0.005 | 0.019 | 3.951 |
| Edge Sealing | | | | | |

Melt blown PP of various densities and thicknesses were calendered at a close nip under high pressure to produce a film structure. See test data below (Table 16) to see the various structures created and the performance difference between “calendered” and “uncalendered.”

The 33 gsm melt blown PP was calendered at 210° F., at 10 fpm (feet per minute), at 0.001″ gap, under 1000 psi, to create “PP Film 1”; see Table 16.

Table 16 is shown below:

TABLE 16

| | Tensile Strength | Apparent |
|---|---|---|
| | (ASTM D5035), in/lbs | Elongation (%) |
| PP Film 1-Un-Calendered | 1.253 | 29.30 |
| PP Film 1-Calendered | 2.294 | 15.78 |

A 48 gsm melt spun PP was calendered at 250° F., at 10 fpm, at 0.005″ gap, under 1,000 psi, to create “PP Film 2,” see, Table 17.

Table 17 is shown below:

TABLE 17

| | Tensile Strength | Apparent |
|---|---|---|
| | (ASTM D5035), in/lbs | Elongation (%) |
| PP Film 2-Un-Calendered | 1.788 | 23.398 |
| PP Film 2-Calendered | 3.789 | 8.475 |

A generic SMS polypropylene (PP) material (Green Bay Nonwovens; Green Bay, Wis.) can also be utilized in this and the aforementioned experiment. Many suitable spunbond webs are available for use as a secondary layer in the present invention in view of the teaching provided in this specification (e.g., PP, PET or PLA polymers with hydrophilic or hydrophobic finishes). In the invention, an 18-gsm and 60-gsm SMS web (spunbond/meltblown/spunbond) from Green Bay Nonwovens (Green Bay, Wis.) was evaluated. This is a commodity product used in infant disposable diapers and has a hydrophilic finish. It is very strong and homogeneous of its lightweight and density. The method of construction was identical to the method described above for the PLA material.

Table 18 below shows the mechanical properties of the SMS web tested.

TABLE 18

| | Tensile Strength | Apparent Elongation |
|---|---|---|
| | (ASTM D5035), in/lbs | (%) |
| SMS-18 gsm | 4.598 | 36.254 |
| SMS-60 gsm | 8.149 | 29.931 |

### Example 11

### Preparation and Testing for Biopolymer Gel Cast

The sample preparation and test methods, in creating the medical dressing, are as documented below.

Samples were prepared using the standard procedure as follows and with exceptions noted. An aqueous solution is prepared by first adding all solids together (with the exception of the pH modifier); gel-forming biopolymer, bubble forming agent, and gelling agent. The solids are mixed thoroughly to ensure homogeneity, and set aside. An aqueous solution having been created with the following; deionized (DI) water, water soluble plasticizers, a non-ionic surfactant, and blended for 30 seconds with a handheld homogenizer, to ensure uniformity. The above solids are slowly added to the aqueous solution while blending with the handheld homogenizer; the solution is blended for 5 minutes once all solids have been added. The resulting biopolymer solution is then covered with a breathable material and placed at room temperature (68-72° F.) and allowed to settle for 16-18 hours, enabling suspended air to dissipate from the solution.

After the viscosity and temperature of the solution is recorded for quality control purposes, the biopolymer solution is administered to the hopper of the die cast machine. Before the machine is started, a solution containing pH modifier and DI water is mixed by vigorously shaking, in a capped container. The pH modifier solution is dispensed into the pressure pot. The compressed nitrogen, hooked to the pressure pot is set to about 0-50 lbs. The machine started by switching on the mixer and peristaltic pump motors on the motor switchboard. Once the machine is started, the biopolymer solution is pumped at a rate of about 150-450 g/min, from the hopper, through the nitrogen injection port, by a peristaltic pump (Baldor Industrial Motors) equipped with a 38″ polyester polyurethane tube (ID-0.250″, OD-0.438″, Wall-0.094″). The nitrogen flow-rate through the injection port is maintained at about 400-700 ml/min (metered by a Cole Parmer air flow-meter (0-800 ml/min)). The solution empties into the mixer from the peristaltic tubing. The residence time of the solution in the mixer corresponds to the flow-rate of the biopolymer solution. The pH modifier is introduced to the solution through a port connected directly to the mixer, at a rate of about 15-30 ml/min (metered by a Cole Parmer liquid flow-meter (10-100 ml/min)). The blended biopolymer solution containing pH modifier is then pumped through a 32″ hose to the die head, attached to a rotating bar at the head of conveyor belt, set to a speed of about 1.5-5.5 ft/min. The standard die head used has a feed width of about 4-12″ and about 0.2-1″ thickness. The biopolymer mix exiting the die head has a width of about 4-12 inches. The biopolymer is cast to release paper. Once the process is complete, the release paper with biopolymer cast is removed from the belt and placed on a drying rack system, where it is allowed to cure at room temperature for upwards of 72 hours before testing.

Density:

The density of the dry biopolymer gel cast is determined by the weight of a 5.08 cm by 5.08 cm sample 48-72 hours post cast.

Absorbency:

Absorbency testing of the prototypes was conducted according to SMTL TM-366. A 5.08 cm. by 5.08 cm. sample is cut from the cross-linked, biopolymer gel cast that has been allowed to cure/dry for <24 hours at 68-72° F. The dry weight (g) is measured using an analytical balance and the thickness (cm) is recorded by a digital thickness gauge. The gel cast sample is then placed in an open container with 500 ml of 18 M ohm deionized water (22° C.). The cast is allowed to soak in the water bath for 60+2 minutes. The cast is then placed on a metal grate angled to 45° and allowed to drain for 5(±1) minutes, and is then re-weighed to obtain the saturated weight of gel cast. The absorbency is calculated in the following ways; absorbency coefficient (g/g) (Eq.1.), amount of water held (g) (Eq.2.), and absorbency (g/100 cm2) (Eq.3.).

Eq.1. Absorbency Coefficient: saturated cast (g)/dry cast (g)

Eq. 2. Amount of Water Held: saturated cast (g)-dry cast (g)

Eq. 3. Absorbency (g/100 cm2): by convention for absorbent wound dressings

Lamination:

The lamination of the gel cast to various substrates was assessed after saturation in a water bath during absorbency testing and graded as full lamination (FL), partial lamination (PL), or no lamination (NL).

### Example 12

### Casting Biopolymer Layer to PLA Non-Woven Layer

In one embodiment of the current invention, the biopolymer gel, containing Type A HPMC, is cast to 36, 48, and 70 gsm poly-lactic acid (PLA) un-calendered non-woven fabric.

The un-calendered non-woven fabric is of the exemplification above. The belt speed for the 70 gsm, 48 gsm, and 36 gsm samples are as follows; 40, 60, and 80 ft/min, respectively.

Following the general procedure, the biopolymer gel cast was prepared with the following formulation using the apparatus described in FIG. 1; about 2-5% sodium alginate, about 1-5% HPMC, about 0.2-0.8% calcium carbonate, about 2-5% glycerin, about 6-10% sorbitol, about 0.2-0.8% Tween 20, about 80-88% DI water, and about 1-4% GDL. The prototypes presented in Table 19 were tested for absorbency and lamination to the substrate after 72 hours of curing. The absorbencies of the gel casts on the given substrates were compared to a gel that was cast to release paper. “PL” denotes partial lamination (and hence at risk of the layers decoupling from each other) and “FL” denotes full lamination (and hence, all layers are adhered and fully bound to each other).

The unique design of the gel cast machine aids in process uniformity and repeatability. The outlet of the hopper is strategically placed 1-6 inches above the inlet of the peristaltic pump to create less stress for the pump on the draw by utilizing gravitational force. The nitrogen injector, attached to the base of the hopper, is made of acrylic with a nitrogen inlet port angled in a downward position to allow the nitrogen to flow with the alginate. The nitrogen injector is purposely placed before the peristaltic pump to ensure uniformity of nitrogen content within the alginate mixture, at the same time there is less pressure being introduced into the mixer which allows consistent flow of the GDL. The peristaltic tubing, being 28-34″ in length, allows flexibility for adjusting the alginate flow-rate by manipulating the length of the tube between the hopper and the pump. The GDL is introduced directly into the mixer, to assure homogenous blending of the GDL with the alginate solution. The alginate GDL solution then exits the mixer and travels through a 34-42″ tube to the die head.

Table 19 is shown below:

TABLE 19

| | Thick- | Water | Absorbency | | |
|---|---|---|---|---|---|
| | ness | Held | Coefficient | Absorbency | Lami- |
| Substrate | (cm) | (g) | (g/g) | (g/100 cm²) | nation |
| Release | 0.2667 | 6.939 | 11.69 | 26.89 | — |
| Paper | | | | | |
| 70 gsm PLA | 0.2972 | 8.859 | 12.56 | 34.72 | PL |
| 48 gsm PLA | 0.2819 | 7.480 | 11.00 | 28.99 | PL |
| 36 gsm PLA | 0.2743 | 7.234 | 10.71 | 28.03 | PL |

The gel cast, as a single layer, partially adhered to all versions of the PLA fabric. The partial lamination may have been due to inconsistencies in the PLA fabric or to the lack of hydrophilicity of the fabric. The gel cast on the heaviest PLA (70 gsm) coincidentally afforded the highest average absorbency of 34.72 g/100 cm2 and although it was observed that the absorbencies decreased as the weight of the PLA decreased, absorbency is a function of the thickness of the gelcast layer. Not unexpectedly, the flexibility and conformability of the dressing decreased as the weight of the PLA increased.

### Example 13

### Method for Casting Biopolymer to Polypropylene Non-Woven

In one embodiment of the current invention, the biopolymer gel, containing AnyCoat AN15 HPMC, is cast to 18 gsm and 60 gsm spun-melt-spun (SMS) polypropylene (PP) fabric.

Following the general procedure, the biopolymer gel cast was prepared with the following formulation; about 2-5% sodium alginate, about 1-5%, HPMC, about 0.2-0.8% calcium carbonate, about 2-5% glycerin, about 6-10% sorbitol, about 0.2-0.8% Tween 20, about 80-88% DI water, and about 1-4% GDL. The biopolymer gel was processed by known processes and the nitrogen flow-rate was adjusted to about 500-700 ml/min. The prototypes presented in Table 20 were tested for absorbency and lamination to the substrate after 72 hours of curing. The absorbencies of the gel casts on the given substrates were compared to gel that was cast to release paper.

Table 20 is shown below:

TABLE 20

| | | | Absorbency | | |
|---|---|---|---|---|---|
| | Thickness | Water | Coefficient | Absorbency | Lami- |
| Substrate | (cm) | Held (g) | (g/g) | (g/100 cm²) | nation |
| Release | 0.2667 | 6.939 | 11.69 | 26.89 | — |
| Paper | | | | | |
| 60 gsm PP | 0.2794 | 8.166 | 11.36 | 31.64 | FL |
| SMS | | | | | |
| 18 gsm PP | 0.2769 | 9.211 | 13.88 | 35.69 | FL |
| SMS | | | | | |

The gel cast, as a single layer, fully adhered to all versions of the SMS fabric. The adherence of the gel cast to the SMS material may be due in part to the hydrophilic nature of the fabric. The gel cast on 18 gsm SMS PP fabric afforded the highest average absorbency of 35.69 g/100 cm2. The absorbencies of the gel cast backed with the SMS PP material obtained a greater absorbency than that of the free gel cast; SMS material is hydrophilic and is itself somewhat absorbent but to a much smaller extent than the gel cast biopolymer. Still the absorbency will be primarily dependent on the thickness of the gel cast layer and not necessarily on the thickness of the composite structure as in Example 12.

### Example 14

### Modification of Example 13 Replacing HPMC with Absorbent Thermal Sensitive Material

In one embodiment of the current invention, the biopolymer gel, containing HPC, is cast to 60 gsm spun-melt-spun (SMS) polypropylene (PP) fabric.

Following the general procedure, the biopolymer gel cast was prepared with the following formulation; 2-5% sodium alginate, 1-5%, HPC, 0.2-0.8% calcium carbonate, 2-5% glycerin, 6-10% sorbitol, 0.2-0.8% Tween 20, 80-88% DI water, and 1-4% GDL. The biopolymer gel was processed by standard means and the nitrogen flow-rate was adjusted to 500-700 ml/min.

The HPC compound differs from HPMC with respect to its ability to hold water. At room temperature the HPC compound is hydrophilic, absorbing and tightly holds water, similar to HPMC. However when HPC is in contact with the skin or at any temperature greater than or equal to 37° C., it will become hydrophobic and release moisture in a sustained and controlled manner at the point of contact. This feature is desirable for contact burn wound dressings and low to moderately exudating wounds. At the body's temperature, HPMC continues to absorb and hold fluids making this absorbent the best choice for highly exudating wounds. HPC is a direct replacement (g/g) for HPMC in all of the formulations exemplified herein.

### Example 15

### Casting Biopolymer Dual-Sided Layer to PLA Non-Woven Layer

In one embodiment of the current invention, the biopolymer gel, containing HPMC, was cast to the reverse side of 36, 48, and 70 gsm polylactic acid (PLA) un-calendered, non-woven fabric that had previously been cast upon, shown in the embodiment of Example 12, creating a dual-sided gel cast with a PLA core.

The composition and process settings of the PLA are shown in Example 12.

Following the general procedure, the biopolymer gel cast was prepared with the following formulation; about 2-5% sodium alginate, about 1-5%, HPMC, about 0.2-0.8% calcium carbonate, about 2-5% glycerin, about 6-10% sorbitol, about 0.2-0.8% Tween 20, about 80-88% DI water, and about 1-4% GDL. The biopolymer gel was processed using the standard procedure. The prototypes presented in Table 21 were tested for absorbency and lamination to the substrate after 72 hours of curing. The absorbencies of the gel casts on the given substrates were compared to gel that was cast to release paper.

Table 21 is shown below:

TABLE 21

| | | | Absorbency | | |
|---|---|---|---|---|---|
| | Thickness | Water | Coefficient | Absorbency | Lami- |
| Substrate | (cm) | Held (g) | (g/g) | (g/100 cm²) | nation |
| Release | 0.2667 | 6.939 | 11.69 | 26.89 | — |
| Paper (1^{st} | | | | | |
| Side)* | | | | | |
| Release | 0.2794 | 8.515 | 20.59 | 32.99 | — |
| Paper (2^{nd} | | | | | |
| Side)* | | | | | |
| 70 gsm PLA | 0.4394 | 13.243 | 12.19 | 51.35 | FL |
| 48 gsm PLA | 0.4039 | 14.224 | 13.68 | 55.12 | FL |
| 36 gsm PLA | 0.3886 | 12.109 | 11.97 | 46.92 | FL |

*Release Paper 1st Side = First to be cast Release Paper 2nd Side = Second to be cast

The two gel cast layers, as a dual sided dressing with PLA core, fully adhered to all variations of PLA. The average absorbencies of the dual sided gel cast on PLA ranged from 46.92-55.12 g/100 cm2. With the addition of the second side gel cast, the absorbencies increased by 48-90% when compared to the embodiment of Example 12. The lighter weight PLA constructed dressing had an increased range of flexibility compared to that of the heavier PLA.

### Example 16

### Casting Biopolymer Dual-Sided Layer to Polypropylene Non-Woven Layer

In one embodiment of the current invention, the biopolymer gel, containing HPMC, is cast to the reverse side of 18 gsm and 60 gsm spun-melt-spun (SMS) polypropylene (PP) fabric that had previously been cast upon, shown in the embodiment of Example 13, creating a dual-side gel cast with a SMS PP core.

Following the general procedure, the biopolymer gel cast was prepared with the following formulation about 2-5% sodium alginate, about 1-5%, HPMC, about 0.2-0.8% calcium carbonate, about 2-5% glycerin, about 6-10% sorbitol, about 0.2-0.8% Tween 20, about 80-88% DI water, and about 1-4% GDL. The prototypes presented in Table 22 were tested for absorbency and lamination to the substrate after 72 hours of curing. The absorbencies of the gel casts on the given substrates were compared to gel that was cast to release paper.

Table 22 is shown below:

TABLE 22

| | | | Absorbency | | |
|---|---|---|---|---|---|
| | Thickness | Water | Coefficient | Absorbency | Lami- |
| Substrate | (cm) | Held (g) | (g/g) | (g/100 cm²) | nation |
| Release | 0.2667 | 6.939 | 11.69 | 26.89 | — |
| Paper (1^{st} | | | | | |
| Side) | | | | | |
| Release | 0.2794 | 8.515 | 20.59 | 32.99 | — |
| Paper (2^{nd} | | | | | |
| Side) | | | | | |
| 60 gsm SMS | 0.4877 | 14.535 | 13.99 | 64.44 | FL |
| 18 gsm PLA | 0.3785 | 9.608 | 13.41 | 52.12 | FL |

The two gel cast layers, as a dual sided dressing with an SMS core, fully adhered to both variations of SMS. The average absorbencies for the 18 gsm and 60 gsm SMS PP were 64.44 and 52.12 g/100 cm2, respectively. The inconsistency could be due in part to the difference in average thickness between the two variations. With the addition of the second side of gel cast, the absorbencies were 47%-103% greater than the single sided prototypes explained in Example 13. The hydrophilic nature of the SMS PP core increases the dressings' ability to wick aqueous solutions through the dressing, allowing for rapid absorption of fluids. The SMS material increases the conformability of the dressing compared with the embodiment in Example 15.

### Example 17

### Casting Biopolymer to Alginate Fiber Non-Woven

In one embodiment of the current invention, the biopolymer gel, containing HPMC, is cast to the 100 gsm needle punched alginate fabric (N-100) acquired from Specialty Fibres and Materials Ltd (Coventry, UK).

Following the general procedure, the biopolymer gel cast was prepared with the following formulation; about 2-5% sodium alginate, about 1-5%, HPMC, about 0.2-0.8% calcium carbonate, about 2-5% glycerin, about 6-10% sorbitol, about 0.2-0.8% Tween 20, about 80-88% DI water, and about 1-4% GDL. The biopolymer gel was processed according to our standard procedure with the nitrogen flow-rate adjusted to 500-700 ml/min and a belt speed set to 5-8 ft/min. The die head used in this process was 8″ wide by ⅛″ thick. The prototypes presented in Table 23 were tested for absorbency and lamination to the substrate after 48 hours of curing. The absorbencies of the gel casts on the given substrates were compared to gel that was cast to release paper.

Table 23 is shown below:

TABLE 23

| | | | Absorbency | | |
|---|---|---|---|---|---|
| | Thickness | Water | Coefficient | Absorbency | Lami- |
| Substrate | (cm) | Held (g) | (g/g) | (g/100 cm²) | nation |
| Release | 0.1168 | 1.596 | 3.708 | 6.183 | — |
| Paper | | | | | |
| N-100 | 0.2769 | 5.83 | 11.67 | 22.59 | FL |

The gel cast, layered on fabric made of alginate fibers, fully adhered to the fabric. In addition to similar chemistry, the stacked nature of the fibers of the alginate fabric seem to form a tortuous path of channels allowing the gel cast to penetrate further affording a tight mechanical bond. The absorbency of this prototype in comparison with the free gel cast was 3.7 times greater. The absorbencies of the gel cast layered alginate fabric and the free gel cast were 22.59 and 6.18 g/100 cm2, respectively.

### Example 18

### Casting Biopolymer Layer to Alginate Non-Woven Layer with Calendered PLA

In one embodiment of the current invention, the biopolymer gel, containing HPMC, is cast to the 100 gsm alginate fabric acquired from Specialty Fibres and Materials Ltd (Coventry, UK) needle punched to a 33 gsm calendered non-woven PLA backing using industry standard methodology.

Following the general procedure, the biopolymer gel cast was prepared with the following formulation; about 2-5% sodium alginate, about 1-5%, HPMC, about 0.2-0.8% calcium carbonate, about 2-5% glycerin, about 6-10% sorbitol, about 0.2-0.8% Tween 20, about 80-88% DI water, and about 1-4% GDL. The biopolymer gel was processed using the general procedure. The die head used in this process was 8″ wide by ⅛″ thick. The prototypes presented in Table 24 were tested for absorbency and lamination to the substrate after 48 hours of curing. The absorbencies of the gel casts on the given substrates were compared to gel that was cast to release paper.

Table 24 is shown below:

TABLE 24

| | | | Absorbency | | |
|---|---|---|---|---|---|
| | Thickness | Water | Coefficient | Absorbency | Lami- |
| Substrate | (cm) | Held (g) | (g/g) | (g/100 cm²) | nation |
| Release | 0.1168 | 1.596 | 3.708 | 6.183 | — |
| Paper | | | | | |
| 33 gsm PLA | 0.2896 | 6.863 | 11.677 | 26.593 | FL |
| N-100 | | | | | |

The Biopolymer gel cast of Example 18 herein fully adhered to the alginate fabric. The absorbency capacity of the product, when compared to the embodiment of Example 16, was 18% greater with an absorbency of 26.59 g/100 cm². The added PLA barrier afforded enhanced structural integrity than that of Example 16.

### Example 19

### Casting Biopolymer to Alginate Non-Woven with Calendered PLA

In one embodiment of the current invention, the biopolymer gel, containing HPMC, is cast to the 100 gsm alginate fabric needle punched to an un-calendered non-woven 55 gsm PLA backing. The un-calendered PLA non-woven backing, manufactured as exemplified above with a belt-speed of 50 ft/min.

Following the general procedure, the biopolymer gel cast was prepared with the following formulation; about 2-5% sodium alginate, about 1-5%, HPMC, about 0.2-0.8% calcium carbonate, about 2-5% glycerin, about 6-10% sorbitol, about 0.2-0.8% Tween 20, about 80-88% DI water, and about 1-4% GDL. The biopolymer gel was processed using the general procedure and an adjusted nitrogen flow-rate of 500-700 ml/min and a belt speed of 5-8 ft/min. The die head used in this process was 8″ wide by ⅛″ thick. The prototypes presented in Table 25 were tested for absorbency and lamination to the substrate after 48 hours of curing. The absorbencies of the gel casts on the given substrates were compared to the gel that was cast onto release paper.

Table 25 is shown below:

TABLE 25

| | | | Absorbency | | |
|---|---|---|---|---|---|
| | Thickness | Water | Coefficient | Absorbency | Lami- |
| Substrate | (cm) | Held (g) | (g/g) | (g/100 cm²) | nation |
| Release | 0.1168 | 1.596 | 3.708 | 6.183 | — |
| Paper | | | | | |
| 55 gsm PLS | 0.1575 | 7.359 | 13.286 | 28.517 | FL |
| N-100 | | | | | |

The gel cast, layered on needle punched alginate fabric that had been cast to 55 gsm PLA, fully adhered to the alginate fabric as explained in Example 16. The heavier PLA made the prototype more rigid than that of the prototype in Example 17 with the 33 gsm PLA. The average absorbency obtained was 28.52 g/100 cm², 7% greater than that of the prototype in Example 6 and 26.5% greater than that of the prototype without the PLA barrier shown in Example 17.

### Example 20

### Casting Biopolymer to PLA Non-Woven with Additional Process Parameters

In one embodiment of the current invention, the biopolymer gel, containing HPMC, was processed with nitrogen flow-rates of 500 ml/min and 600 ml/min in order to obtain a product with the highest absorbency and physical integrity. The gel was cast to 70 gsm PLA in order to determine the effect of the nitrogen flow-rate on the lamination of the gel cast to the substrates. The composition and process conditions for the 70 gsm non-woven PLA are outlined in Example 13 and Table 19.

Following the general procedure, the biopolymer gel cast was prepared with the following formulation; about 2-5% sodium alginate, about 1-5%, HPMC, about 0.2-0.8% calcium carbonate, about 2-5% glycerin, about 6-10% sorbitol, about 0.2-0.8% Tween 20, about 80-88% DI water, and about 1-4% GDL. The biopolymer gel was processed using the general procedure with the exception of varying the nitrogen flow-rate as follows; 500 ml/min and 600 ml/min. The gel casts on the various substrates presented in Table 25 were tested for absorbency and lamination to the substrate after 48 hours of curing.

Table 25 is shown below:

TABLE 25

| | | | Absorbency | | |
|---|---|---|---|---|---|
| | Thickness | Water | Coefficient | Absorbency | Lami- |
| Substrate | (cm) | Held (g) | (g/g) | (g/100 cm²) | nation |
| 70 gsm PLA @ | 0.2921 | 7.828 | 12.1182 | 30.3320 | FL |
| 500 ml/min | | | | | |
| 70 gsm PLA @ | 0.3233 | 8.0098 | 13.9928 | 31.0379 | FL |
| 600 ml/min | | | | | |

Varying the flow-rate of nitrogen did not significantly affect the absorbency or the ability to laminate to the prototype. However, the integrity of the gel cast was impacted between the flow-rates. The gel cast processed at 600 ml/min nitrogen had greater structural integrity measured qualitatively by pinching the product between one's finger tips. The gel cast, processed at 500 ml/min, is broken through when pinched aggressively, whereas the gel cast processed at 600 ml/min, when compressed, does not deteriorate under pressure.

### Example 21

### Casting Biopolymer to SMS PP Non-Woven with Additional Process Parameters

In one embodiment of the current invention, the biopolymer gel, containing HPMC, was processed with nitrogen flow-rates of 500 ml/min and 600 ml/min in order to obtain a product with the highest absorbency and physical integrity. The gel was cast to 60 gsm SMS PP, to determine the effect of the nitrogen flow-rate on the lamination of the gel cast to the substrates.

Following the general procedure, the biopolymer gel cast was prepared with the following formulation; about 2-5% sodium alginate, about 1-5% HPMC, about 0.2-0.8% calcium carbonate, about 2-5% glycerin, about 6-10% sorbitol, about 0.2-0.8% Tween 20, about 80-88% DI water, and about 1-4% GDL. The biopolymer gel was processed using the general procedure with the exception of varying the nitrogen flow-rate as follows; 500 ml/min and 600 ml/min. The gel casts on the various substrates presented in Table 26 were tested for absorbency and lamination to the substrate after 48 hours of curing.

Table 26 is shown below:

TABLE 26

| | | | Absorbency | | |
|---|---|---|---|---|---|
| | Thickness | Water | Coefficient | Absorbency | Lami- |
| Substrate | (cm) | Held (g) | (g/g) | (g/100 cm²) | nation |
| 60 gsm SMS @ | 0.3327 | 8.271 | 12.8517 | 32.0492 | FL |
| 500 ml/min | | | | | |
| 60 gsm SMS @ | 0.2979 | 6.965 | 9.3804 | 26.9885 | FL |
| 600 ml/min | | | | | |

The results of this example are parallel to that of the embodiment of Example 19. The difference seen in average absorbency values, between the two flow-rates, is likely due to the variance in the average thickness of the gel cast.

### Example 22

### Casting Biopolymer to SMS PP Non-Woven with Additional Process Parameters

In one embodiment of the current invention, the biopolymer gel, containing HPMC, was processed by varying belt-speeds to obtain a variation of gel cast thicknesses and to test the corresponding absorbencies. The gel was cast to 60 gsm SMS PP and was tested for lamination integrity after saturating with DI water during a 24 hour absorbency analysis.

Following the general procedure, the biopolymer gel cast was prepared with the following formulation; about 2-5% sodium alginate, about 1-5%, HPMC, about 0.2-0.8% calcium carbonate, about 2-5% glycerin, about 6-10% sorbitol, about 0.2-0.8% Tween 20, about 80-88% DI water, and about 1-4% GDL. The biopolymer gel was processed using the general procedure with the exception of varying the belt-speed as follows; 3.5 ft/min, 2.2 ft/min, and 1.5 ft/min. The gel casts presented in Table 27 were subjected to a 24 hour absorbency test, and the lamination to the substrate was assessed, after one week of curing.

Table 27 is shown below:

TABLE 27

| | | | Absor- | | | |
|---|---|---|---|---|---|---|
| | | | bency | Absor- | | |
| | Thick- | Water | Coeffi- | bency | Absor- | Lam- |
| | ness | Held | cient | (g/100 | bency | ina- |
| Belt-speed | (cm) | (g) | (g/g) | cm²) | (g/cm3) | tion |
| 3.5 ft/min | 0.2906 | 9.601 | 15.27 | 37.20 | 1.27 | FL |
| 2.2 ft/min | 0.4285 | 14.305 | 16.02 | 55.43 | 1.23 | FL |
| 1.5 ft/min | 0.5291 | 16.809 | 14.29 | 65.13 | 1.32 | PL |

Reducing the belt-speed results in increasing the thickness of the gel cast and the concomitant increase in the absorbency of the gel cast samples. In Table 27, the absorbency per volume stays consistent (1.2+0.1 g/cm³) among the three belt-speed variations showing that the absorbency increases linearly as a function of gel cast thickness. However, the lamination of the gel cast to the substrate may be the limiting factor in attaining a thickness that will not delaminate from the substrate itself. The gel cast processed at a belt-speed of 1.5 ft/min, with a thickness of 0.5291 cm, partially laminated to the SMS PP material. Thickness of the gel cast could be a factor in the partial delamination of the gel cast from the substrate.

### Example 23

### Active Layer Deposition onto the Biopolymer Gel Cast with Experimental Drying

In one embodiment of the current invention, an active coating (layer 17 from the FIGS. 2-6) is formulated with about 1.0-1.5% of the silver zeolite, about 1-5% collagen, about 0.5-2.00% sodium hyaluronate, about 2.0-5.0% sodium alginate, about 2-5% glycerin, about 1-5% HPMC, about 0.2-0.8% calcium carbonate, about 1-4% GDL and about 6-10% sorbitol in about 80-88% DI water.

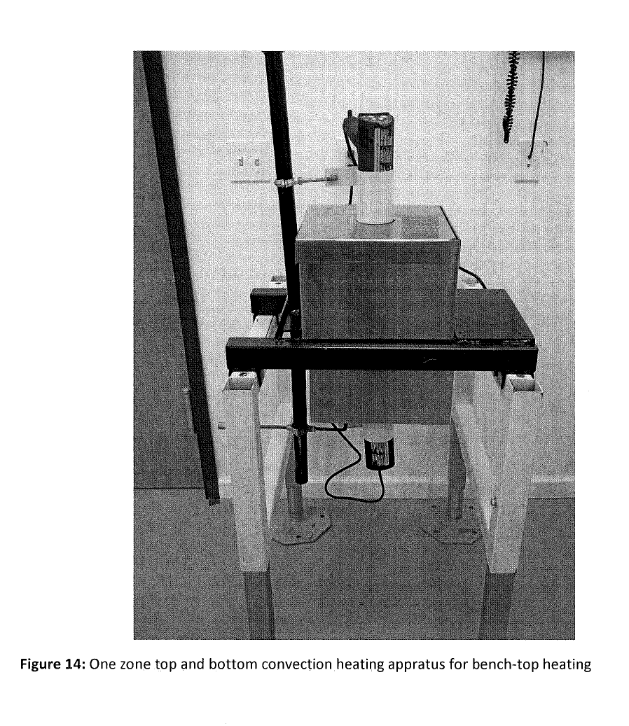

This active coating is cast onto the cross-linked biopolymer gelled composite which was produced according to the general procedure described herein. The coated gelled composite is air treated with mild top and bottom one zone convection heating (or with low percentage IR heating) using an apparatus shown in FIG. 14. The heating apparatus is composed of a single compartment with a metal grate in the center of the oven to place the samples. The oven is heated with two Milwaukee Model MHT3300 1500 watt heat (Brookfield, Wis.) guns (above and below the metal grate) with a temperature range from 250-1350° F.; with high and low speed settings. The heat guns attached to a metal beam are adjustable creating a distance range of 5.5″ to 10″ from the metal grate.

### Example 24

### Active Layer Deposition on the Biopolymer Cast

In one embodiment of the current invention, an active layer containing collagen, hyaluronan (HA) and a film former was incorporated on the surface of the cross-linked, biopolymer gel cast composite.

The gel cast was created following the general procedure, the biopolymer gel cast was prepared with the following formulation; about 2-5% sodium alginate, about 1-5%, HPMC, about 0.2-0.8% calcium carbonate, about 2-5% glycerin, about 6-10% sorbitol, about 0.2-0.8% Tween 20, about 80-88% DI water, and about 1-4% GDL. The biopolymer gel was processed using the general procedure.

This embodiment of the invention is intended to be merely exemplary; numerous variations and modifications will be apparent to those skilled in the art. All such variations and modifications are intended to be within the scope of the present invention as defined in any appended claims.

### Example 25

### Active Layer Deposition on the Biopolymer Cast Layer—2

In one embodiment of the current invention, an active coating (layer 17 from the FIGS. 2-6) is formulated with: 0.5-8% of X-static®, 1-5% collagen, 0.5-2% sodium hyaluronate, 1-5% sodium alginate, 2-5% glycerin, 6-10% sorbitol, 0.2-0.8% Tween 20, 0.2-0.8% calcium carbonate, 1-5% HPC, and 1-4% GDL in about 80-88% DI water. This active coating is cast onto the cross-linked biopolymer gelled composite, immediately after it is cast, which was produced according to the general procedure detailed in Example 11. The active coat was applied to the gel cast within a 15-120 minute window in order to obtain maximum crosslinking between the two layers.

The antimicrobial efficacy was obtained by NAMSA (Irvine, Calif.) for the above formulation. Table 28 documents the North American Science Associates (NAMSA; Northwood, Ohio) results for the following organisms: Methicillin resistant Staphylococcus aureus (MRSA) source no. ATCC 33591, Klebsiella pneumoniae source no. ATCC 4352 Pseudomonas aeruginosa source no. ATCC 9027, Candida albicans source no. ATCC 10231, Vancomycin resistant Enterococcus (VRE) source no. ATCC 51575 and Acinetobacter baumannii source no. ATCC 19606.

TABLE 28

| | Organism Count | Organism Count | |
|---|---|---|---|
| Organism | (CFU/mL)-Zero | (CFU/mL)-4 | Percent |
| Identification | Time | Hour | Reduction |
| MRSA | 2.0 × 10⁶ | 7.50 × 10² | 99.97 |
| K. pneumoniae | 1.25 × 10⁶ | 1.0 × 10² | >99.99 |
| P. aeruginosa | 1.25 × 10⁶ | <1.0 × 10² | >99.99 |
| A. baumannii | 3.85 × 10⁶ | <1.0 × 10² | >99.99 |
| C. albicans | 1.75 × 10⁶ | <1.0 × 10² | >99.99 |
| VRE | 5.30 × 10⁶ | 2.0 × 10² | >99.99 |

### Example 26

### Active Layer Deposition on the Biopolymer Gel Cast Layer—Ag

In another embodiment of the current invention, an active coating (layer 17 from the FIGS. 2-6) is formulated with: 0.5-8% of Agion® Silver Zeolite, 1-5% collagen, 0.5-2% sodium hyaluronate, 1-5% sodium alginate, 2-5% glycerin, 6-10% sorbitol, 0.2-0.8% Tween 20, 0.2-0.8% calcium carbonate, 1-5% HPC, and 1-4% GDL in about 80-88% DI water.

This active coating is cast onto the cross-linked biopolymer gelled composite, immediately after it is cast, which was produced according to the general procedure detailed in Example 11. The active coat was applied to the gel cast within a 15-120 minute window in order to obtain maximum crosslinking between the two layers.

The antimicrobial efficacy was obtained by NAMSA (Irvine, Calif.) for the above formulation. Table 29 documents the NAMSA results for the following organisms: Methicillin resistant Staphylococcus aureus (MRSA) source no. ATCC 33591, Pseudomonas aeruginosa (PA) source no. ATCC 9027, and Acinetobacter baumannii (AB) source no. ATCC 19606.

TABLE 29

| Test Article | Organism Count | Organism Count | Percent |
|---|---|---|---|
| Identification | (CFU/mL)-Zero Time | (CFU/mL)-Hour | Reduction |
| MRSA | 2.75 × 10⁶ | 1.95 × 10⁵ | 94.40 |
| P. aeruginosa | 1.41 × 10⁶ | 3.35 × 10³ | 99.87 |
| A. baumannii | 5.05 × 10⁶ | 6.00 × 10² | 99.99 |

### Example 27

### Active Layer Deposition on the Biopolymer Cast Layer—Ag

In one embodiment of the current invention, an active coating (layer 17 from the FIGS. 2-6) is formulated with: 0.5-8% of Ionpure® WPA Silver Zeolite, 1-5% collagen, 0.5-2% sodium hyaluronate, 1-5% sodium alginate, 2-5% glycerin, 6-10% sorbitol, 0.2-0.8% Tween 20, 0.2-0.8% calcium carbonate, 1-5% HPC, and 1-4% GDL in about 80-88% DI water.

This active coating is cast onto the cross-linked biopolymer gelled composite, immediately after it is cast, which was produced according to the general procedure detailed in Example 11. The active coat was applied to the gel cast within a 15-120 minute window in order to obtain maximum crosslinking between the two layers.

### Example 28

### Differentiation Between Gel Cast and Foam

Although this bio-polymeric gel composite dressing would have similar indications for use as a foam dressing, their physical structures are very different.

To test and demonstrate the difference, wet gel cast material was manufactured as exemplified above with a thickness of 0.317 cm. Commercial medical grade Rynel foam (Wiscasset, Me.) at a thickness of 0.488 cm was acquired and used for the purpose of comparison.

Both the gel cast and the foam material was cut into a sheet of 6 in by 6 inches. Both materials were then slowly and gently placed in a pan of water, for one hour before any assessments were made, with the function of water to simulate wound exudates in a wound bed.

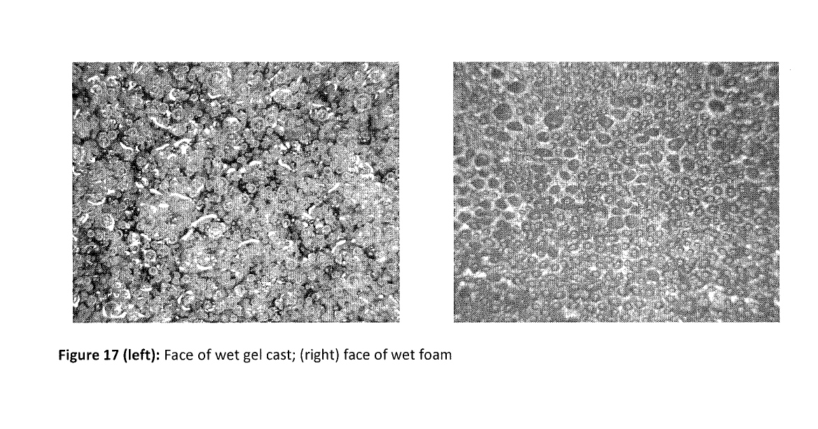

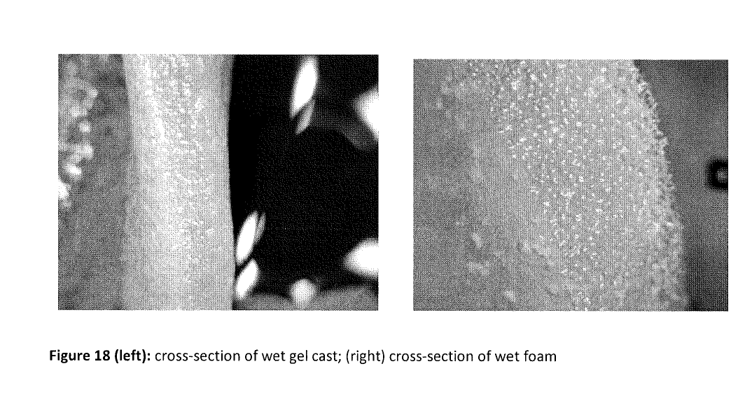

It is clear from the pictures (see FIG. 17, right) of the “face” of the wet structures that the foam is a continuous polymeric structure which forms regular open cells, whereas the gel cast material (see FIG. 17, left) has no continuous polymeric structure but rather appears to be a discontinuous phase randomly dispersed in a continuous phase. By way of analogy, they are no more similar than a “crystalline” structure (rigid, discreet domains) vs. an “amorphous” structure (comingled, non-discreet domains). In fact, upon closer inspection, the gel cast material seems to be comprised of agglomerated individual gelled (cured) bubbles with interstices (FIG. 18, left). These cured, intact bubbles are held together by a combination of forces such as hydrogen bonding and/or van der Waals in contrast to the rigid cells formed within the polymer resin during the foaming process (FIG. 18, right).

Upon examination, the dry gel cast product was smooth, and “silky” to the touch.

When cut with scissors, the edges were smooth and regular and did not fray or generate debris.

The gel cast does not have a cellular structure but rather is comprised of individual, cured, intact bio-polymeric bubbles held together to form voids or interstices among the agglomerates.

Upon exposure to water, the gel cast wicks quickly to complete saturation, is smooth and lubricious to the touch, has very high absorbency, and holds the water tightly even in a vertical position. “Holding the water tightly” is defined as minimal or trivial amount of water, less than 0.01 gram, flowing out of the material.

Further, when compression is applied to the saturated gel cast, by simply and gently pressing the material between one's fingers, it very quickly wicks water back to complete saturation immediately upon removal of the compression. Complete saturation is defined as the re-attainment of the original material thickness together with visual verification of no unsaturated areas in the material.

In addition, upon compression the intact bio-polymeric bubbles themselves do not compress but rather yield to the compression by moving away from the source, returning quickly to fill the void created by removal of the source of compression.

Upon examination, the prior art dry medical foam product was less “silky” to the touch and somewhat less rigid than the gel cast material of the present invention.

When cut with scissors, the edges of the prior art foam are rough, irregular, and tend to fray and/or leave debris behind.

The prior art foam has a regular, well defined, continuous, cured polymeric cellular structure formed by the introduction of a gas which escapes post cure.

When introduced into water, the prior art foam wicks more slowly to complete saturation, is somewhat rougher and significantly less lubricious to the touch, is highly absorbent (albeit less so than the gel cast of the present invention), and did not to hold tightly to the water especially when placed in a vertical position—it drains nearly completely and very quickly.

When compression is applied to the water saturated prior art foam and upon the removal of that compression, the foam's cellular structure is physically deformed, does not recover quickly and does not wick water back to its original saturation level.

### Example 29

### Active Layer Deposition on Negative Pressure Wound Therapy Foam

This example illustrates active layer deposition on negative pressure wound therapy foam (FIG. 19). The active coat is composed of about 1-5% sodium alginate, about 1-5% hydroxypropyl cellulose, about 0.2 0.8% calcium carbonate, about 6-10% sorbitol, about 2-5% glycerin, about 1-4% glucono delta lactone, about 0.1-0.8% Tween 20, about 0.5-2% hyaluronan (HA), about 0.25-2% silver zeolite, about 0.5-5% collagen, and about 80-88% deionized water.

The active coat (17) is extruded onto negative pressure wound therapy foam (21) using the gel cast aeration apparatus illustrated in FIG. 19. The active coat is processed using a ⅔″ slit die head with the standard process flow-rates. The resulting layer is 0.2-0.4″ in thickness.

### Example 30

### Application of Active Layer by Dipping onto Negative Pressure Wound Therapy Foam

This example illustrates the application of a low solids, alginate based active coat (17) by dipping, spraying or printing onto the Negative Pressure Wound Therapy foam (21), coating the interior and exterior surfaces of the cellular foam without occluding the cells (FIG. 20). The active coat is composed of about 1-5% sodium alginate, about 0.2-0.8% calcium carbonate, about 6-10% sorbitol, about 1-5% glycerin, about 1-4% glucono delta lactone, about 0.5-2% hyaluronan (HA), 1-8% X-Static, about 1-5% collagen, and about 80-88% deionized water.

What is claimed is:

1. A medical dressing comprising a biopolymer layered structure, the biopolymer layered structure comprising:
a biodegradable, bioresorbable layer comprising a plurality of biodegradable, bioresorbable fibers, wherein the fibers are oriented to provide compression resistance and maintain paths for liquid-flow and air-flow, and a bioresorbable, biodegradable hydrophilic surface coating on a substantial number of the fibers; the fibers incorporating one or more bioactive agents.

2. The medical dressing of claim 1, wherein the layered structure comprises one or more natural fibers selected from the group consisting of cotton, bamboo and sisal.

3. The medical dressing of claim 1, wherein the layered structure comprises one or more fibers manufactured from natural sources selected from the group consisting of polylactide, polyglycolide, poly-L-lactide, poly-DL-lactide, polycaprolactone, polyhydroxyalkanoate, viscose, polyethylene terephthalate and polypropylene.

4. The medical dressing of claim 3, wherein the fibers comprise polymers of polylactide.

5. The medical dressing of claim 1, wherein the bioresorbable hydrophilic surface coating is on a substantial number of the fibers located proximate to other layers of the medical dressing.

6. The medical dressing of claim 1, wherein each of the fibers in the plurality of fibers has a diameter of approximately 1 μM to 1 mm.

7. The medical dressing of claim 6, wherein each of the fibers in the plurality of fibers has a diameter of approximately 5 to 100 μM.

8. The medical dressing of claim 1, wherein the fibers are processed by one or more of being cut into a staple of selected length, carded, air-layered, needle-punched, vertically lapped, spirally wound, thermally bonded, or ultrasonically bonded.

9. The medical dressing of claim 8 wherein the fibers of the layered structure are vertically lapped or spirally wound.

10. The medical dressing of claim 1, wherein the bioresorbable hydrophilic surface coating comprises one or more of cellulose, alginate, gums, starch, chitosan, ethylene glycol, carrageenans, polyoxethylene and polylactic acid.

11. The medical dressing of claim 10 wherein the bioresorbable hydrophilic surface coating comprises polylactic acid.

12. The medical dressing of claim 10 wherein the bioresorbable hydrophilic surface coating comprises alginate.

13. The medical dressing of claim 1, wherein the bioactive agent is an antimicrobial agent.

14. The medical dressing of claim 13, wherein the antimicrobial bioactive agent comprises asilver species.

15. The medical dressing of claim 13, wherein the bioactive agent is a component of one or more of the fibers and the surface coating.

16. The medical dressing of claim 1, further comprising:
a) a semi-permeable layer over-lying the biopolymer layered structure and extending beyond the biopolymer layered structure to form a peripheral region, that is sealable to the skin of the subject; and
b) a port, coupled to the semi-permeable layer, that is connectable to a negative pressure generating device.

17. The medical dressing of claim 16, further comprising an adhesive layer disposed on the peripheral region, which causes adherence of the semi-permeable layer to the skin.

18. The medical dressing of claim 16, wherein the semi-permeable layer is defined by a moisture-vapor transmission rate of 1 to 1000 g/24 hr-m² (grams per 24 hour-meter squared).

19. The medical dressing of claim 1, where the diameter of the fibers is selected to provide a desired compression resistance between a range of 0% and 50%.

20. A method of treating a wound, the method comprising:
a) providing a wound dressing comprising (i) a bioresorbable biodegradable non-woven material comprising a plurality of bioresorbable fibers incorporating a bioactive agent, the wound dressing having a wound interface for contacting a surface of a wound, wherein the fibers are predominately oriented in a direction transverse to an exposed surface to provide compression resistance and maintain paths for liquid-flow and air-flow, and (ii) a bioresorbable hydrophilic surface coating on a substantial number of the fibers proximal to the wound interface;
b) applying said wound dressing to the wound with the bioresorbable hydrophilic surface in contact with the surface of the wound, thereby protecting the wound by providing resistance to compression, maintaining paths for air-flow and fluid-flow and removing exudate from the wound through one or more of absorption and negative pressure.

21. The method of claim 20, wherein the fibers are natural fibers selected from one or more of the group consisting of cotton, bamboo and sisal.

22. The method of claim 20, wherein the bioresorbable fibers comprise one or more synthetic fibers selected from the group consisting of polylactide, polyglycolide, poly-L-lactide, poly-DL-lactide, polycaprolactone, viscose, PET, and PHA.

23. The method of claim 20, wherein the fibers comprise polymers of polylactide.

24. The method of claim 20, wherein each of the fibers in the plurality of fibers has a diameter of about 1 μm to about 1 mm.

25. The method of claim 20, wherein the fibers are processed by one or more of being cut into staple of selected length, carded, air-layered, needle-punched, vertically lapped, spirally wound or thermally bonded.

26. The method of claim 20, wherein the bioresorbable hydrophilic surface coating comprises but is not limited to one or more of cellulose, alginate, carrageenans, gums, starch, ethylene glycol, poly-oxethylene and polylactic acid.

27. The method of claim 26, wherein the bioresorbable hydrophilic surface coating comprises polylactic acid.

28. The method of claim 26, wherein the bioresorbable hydrophilic surface coating comprises alginate.

29. The method of claim 20, wherein the bioresorbable surface coating wicks exudate from the wound.

30. The method of claim 20, further comprising removal of the exudate from the surface coating by negative pressure.

31. The method of claim 20, wherein a bioactive agent is incorporated into one or more of the fibers and the surface coating.

32. The method of claim 20 wherein the bioactive agent is an antimicrobial agent.

33. The method of claim 32, wherein the antimicrobial bioactive agent comprises a mixture of two or more components selected from a group consisting of (i)—silver ion-exchange particles, (ii) silver in the form of a water-soluble matrix, and (iii) silver in the form of metal coated fibers.

34. The method of claim 20, wherein the exudate is removed by a vacuum.

35. The method of claim 20, wherein the wound dressing is placed into the wound so as to fill 25% or more of the volume of the wound.
